(19) United States
(12) Patent Application Publication
(10) Pub. No.: US 2002/0160495 A1
(43) Pub. Date: Oct. 31, 2002

(54) Soluble ischemia activated protein

(75) Inventors: Oleg Mirochnitchenko, East Burnswick, NJ (US); Jiang Wei, Highland Park, NJ (US); Masayori Inouye, Piscataway, NJ (US)

Correspondence Address: LYON & LYON LLP, 633 WEST FIFTH STREET, SUITE 4700, LOS ANGELES, CA, 90071, US

(73) Assignee: UNIVERSITY OF MEDICINE AND DENTISTRY

(21) Appl. No.: 09/960,631

(22) Filed: Sep. 20, 2001

Related U.S. Application Data
(60) Provisional application No. 60/233,819, filed on Sep. 20, 2000.

(51) Int. Cl. C12N 9/64; C07H 21/04; C12P 21/02; C12N 5/06

(52) U.S. Cl. 435/226; 435/69.1; 435/320.1; 435/455; 435/325; 536/23.2

(57) ABSTRACT

The present invention provides a novel gene, IAP, which encodes a soluble protein that is involved in the initiation of an inflammatory cytokine response and which may interact with the anti-inflammatory IL-13. The protein of the present invention may be involved as a central mediator in ischemia, reperfusion, asthma and other inflammation-induced pathological conditions. The invention relates to methods for using isolated polypeptides and polynucleotides, for detecting the early onset of chronic asthma, psoriasis, stroke, ischemia, reperfusion, leishmaniasis, helminthiasis, hypoxia, or other causes of renal, liver or heart failure in a mammal, where increased cytokine activity is known to play a role. The invention, further, relates to methods and compositions for detecting ischemia, specifically silent ischemia, by obtaining a test sample from a mammal, measuring the level of IAP mRNA or protein in the test sample, and determining if the level of IAP mRNA or protein measured in the test sample correlates with elevated levels indicative of an ischemia associated disease.

The present utility patent application claims priority to provisional patent application U.S. Ser. No. 06/233,819 (Mirochnitchenko et al.), filed Sep. 20, 2000, the disclosure of which is incorporated by reference in its entirety herein.

### FIELD OF THE INVENTION

The present invention relates to the field of molecular biology, ischemia and reperfusion, and the regulation of cytokine activity. In particular, this invention provides a novel gene, IAP (ischemia activated protein), which is found in mouse, rat, bovine and human. IAP plays an important role in the inflammatory cytokine response and is an indicator of the onset of ischemia, reperfusion and hypoxia.

### BACKGROUND OF THE INVENTION

Various publications or patents are referred to in parentheses throughout this application to describe the state of the art to which the invention pertains. Each of these publications or patents is incorporated by reference herein. Complete citations of scientific publications are set forth in the text or at the end of the specification.

Damage occurs to cells in mammals that have been deprived of oxygen. The interruption of blood flow, whether partial (hypoxia) or complete (ischemia), and the ensuing inflammatory responses may be the most important cause of coagulative necrosis or cell death in human disease. The complications of atherosclerosis, for example, are generally the result of ischemic cell injury in the brain, heart, small intestines, kidneys, and lower extremities. Highly differentiated cells, such as the proximal tubular cells of the kidney, liver, cardiac myocytes, and the neurons of the central nervous system, all depend on aerobic respiration to produce ATP, the energy necessary to carry out their specialized functions. When ischemia limits the oxygen supply and ATP is depleted, the affected cells may become irreversibly injured. The ensuing inflammatory responses to this initial injury provide additional insult to the affected tissue. Examples of such hypoxia or ischemia are the partial or total loss of blood supply to the body as a whole, an organ within the body, or a region within an organ, such as occurs in cardiac arrest, pulmonary embolus, renal artery occlusion, coronary occlusion or occlusive stroke, inflammatory kidney or liver diseases, and circumstances involving septic shock or organ transplantation.

Inflammation is the response of tissue to injury. In the acute phase it is characterized by increased blood flow and vascular permeability along with the accumulation of fluid, leukocytes, and inflammatory mediators such as cytokines. In the chronic phase it is characterized by the development of specific humoral and cellular immune responses to pathogens present at the site of tissue injury. During both acute and chronic inflammatory processes, a variety of soluble factors are involved in leukocyte recruitment through increased expression of cellular adhesion molecules and chemo-attraction. These soluble mediators regulate the activation of fibroblasts, endothelial cells, tissue macrophages, and mast cells and help recruit and regulate other inflammatory cells such as monocytes, lymphocytes and neutrophils.

Among the soluble factors that mediate these responses are a group of cell derived polypeptides, known as cytokines, which to a large extent orchestrate the inflammatory response—they are major determinants of the make-up of the cellular infiltrate, the state of cellular activation, and the systemic responses to inflammation. Most cytokines are multifunctional. They are pleiotropic molecules that elicit their effects locally or systemically in an autocrine or paracrine manner. Cytokines are involved in extensive networks that involve synergistic as well as antagonistic interactions and exhibit both negative and positive regulatory effects on various target cells.

The tissue damage associated with ischemia-reperfusion injury is believed to comprise both the initial cell damage induced by the deprivation of oxygen to the cell and its subsequent recirculation, as well as the damage caused by the body's response to this initial damage. The secondary damage, resulting from the inflammatory response, is likely the source of significant tissue damage. It is thought that reperfusion may result in dysfunction to the endothelium of the vasculature as well as injury to the surrounding tissue. Among the factors thought to mediate these damaging effects are those associated with modulating the body's inflammatory response following tissue injury, e.g., cytokines such as IL-1 and tumor necrosis factor (TNF), and oxygen-derived free radicals such as superoxide anions. These humoral agents are produced by adhering neutrophilic leukocytes or by endothelial cells and have been identified at ischemic sites upon reperfusion. Moreover, TNF concentrations are increased in humans after myocardial infarction. The tissue damage associated with hyperoxia injury is believed to follow a similar mechanism, where the initial damage is mediated primarily through the presence of toxic oxygen metabolites.

A dynamic and ever-shifting balance exists between proinflammatory cytokines and anti-inflammatory components of the human immune system. The net effect of any cytokine is dependent on the timing of cytokine release, the local milieu in which it acts, the presence of competing or synergistic elements, cytokine receptor density, and tissue responsiveness to each cytokine.

CD4+ T helper (Th) lymphocytes can differentiate into functionally dichotomous subsets of Th cells depending on the microenvironment of the cell. The cytokine-producing CD4+ helper cells are classified into T_{H1}- and T_{H2}-type cells on the basis of the cytokines produced. T_{H1}-type cells secrete high levels of IL-2, TNF-α, and interferon-γ (FN-γ). This activates macrophages and promotes cell-mediated immune responses against invasive intracellular pathogens. T_{H2}-type cells produce a variety of anti-inflammatory cytokines, including IL-4, IL-5, IL-6, IL-10, and IL-13. Both T_{H1} and T_{H2} cells produce lesser amounts of TNF-α, granulocyte-macrophage colony-stimulating factor (GM-CSF), and IL-3. T_{H2}-type cytokines promote humoral immune responses against extracellular pathogens.

Inflammatory cytokines can be divided into two groups: those involved in acute inflammation (IL-1, TNFalpha, IL-6, IL-8, IL-11 and other chemokines, G-CSF and GM-CSF) and those responsible for chronic inflammation (which can be subdivided into cytokines mediating humoral responses such as IL-4, IL-5, IL-6, IL-7, and IL-13, and those mediating cellular responses such as IL-1, IL-2, IL-3, IL-4, IL-7, IL-9, IL-10, IL-12, interferons, and TNF beta). Some cytokines, such as IL-1, significantly contribute to both acute and chronic inflammation.

IL-1 alpha can trigger fever by enhancing prostaglandin E2 (PGE2) synthesis by the vascular endothelium of the hypothalamus and can stimulate T cell proliferation. In addition, IL-1 elicits the release of histamine from mast cells at the site of inflammation. Histamine then triggers early vasodilation and increase of vascular permeability. The pro-inflammatory effects of IL-1 can be inhibited by IL-1 receptor antagonist (IL-IRa). IL-1Ra is produced by immune complex- or IL-4-stimulated macrophages and by TNF or GM-CSF-stimulated neutrophils.

IL-1 and TNF-alpha share several pro-inflammatory properties. TNF-alpha exists as a dimer and is one of the products of activated macrophages/monocytes, fibroblasts, mast cells, and some T and natural killer cells. Like IL-1, TNF-alpha can induce fever, either directly via stimulation of PGE2 synthesis by the vascular endothelium of the hypothalamus, or indirectly by inducing release of IL-1. Both cytokines can stimulate the production of collagenase and PGE₂. TNF-β, also known as lymphotoxin, is produced by activated T and B lymphocytes. Its properties are similar to those of TNF-alpha and include the induction of apoptosis (programmed cell death) in many types of transformed, virally infected and tumors cells.

Of considerable interest to immunologists and parasitologists is the distinction between two closely related cytokines, IL-4 and IL-13, which mediate the anti-inflammatory response. IL-4 is a highly pleiotropic cytokine that is able to influence Th cell differentiation. Early secretion of IL-4 leads to polarization of Th cell differentiation toward T_{H2}-like cells. T_{H2}-type cells secrete their own IL-4, and subsequent autocrine production of IL-4 supports cell proliferation. IL-4 is a 20-kd glycoprotein produced by mature T_{H2} cells and cells from the mast cell or basophil lineage. It drives T_{H2} responses, mediates the recruitment and activation of mast cells, and stimulates the production of IgE antibodies via the differentiation of B cells into IgE-secreting cells.

IL-4 has marked inhibitory effects on the expression and release of the proinflammatory cytokines. It is able to block or suppress the monocyte-derived cytokines, including IL-1, TNF-α, IL-6, and IL-8. The T_{H2}-cell secretion of IL-4 and IL-10 leads to the suppression of T_{H1} responses by down-regulating the production of macrophage-derived IL-12 and inhibiting the differentiation of T_{H1}-type cells. It stimulates the synthesis of the cytokine inhibitor IL-1ra. IL-4 is able to affect a variety of structural cells. It can potentiate proliferation of vascular endothelium and skin fibroblasts yet decrease proliferation of adult human astrocytes and vascular smooth muscle cells.

IL-13 was previously thought of as an orphan, relative to its better known “twin,” of IL-4. IL-13 is a potent modulator of human monocytes and B-cell development, function and it prevents apoptosis of these cells. It is secreted by activated T_{H2} lymphocytes. It is a 132-amino-acid nonglycosylated protein with a molecular weight of about 10 kd. The human IL-13 gene has been mapped in close proximity to the IL-4 gene along a 4.5-kilobase sequence of DNA on chromosome 5q31. IL-13 shares many, but not all, of its biologic activities with IL-4. IL-13 and IL-4 share a common cellular receptor (IL-4 type I receptor). The major α-helical regions that are essential for their activity are highly homologous. IL-4R and IL-13R complexes share the IL-4Rα chain required for signal transduction. Hence, these two anti-inflammatory cytokines are very similar although they share only 20% to 25% primary amino acid homology.

The principal functional difference between IL-4 and IL-13 lies in their effects on T cells. IL-4 is a dominant mediator of T_{H2} cell differentiation, proliferation, and activity, whereas IL-13 has minimal effects on T-cell function. IL-13 can down-regulate the production of TNF, IL-1, IL-8 and has profound effects on expression of surface molecules on both monocytes and macrophages. It upregulates cell surface expression of β2 integrins, CD23, CD71, CD72 and major histocompatibility complex (MHC) class II antigens and down-regulates CD14. IL-13 also acts as a chemoattractant for monocytes and it contributes to Dendritic Cell (DC) differentiation and the generation and maintenance of the DC compartment.

IL-13 also enhances the production and/or induction of IgE, IgM, IgG, and IgA. Furthermore, IL-13 induces the low-affinity receptor for IgE (CD23), which, through its capacity to focus allergen/IgE complexes to allergen-specific T cells, is thought to enhance allergic responses. IL-13 is sufficient to elicit IgE secretion from human B cells independently of IL-4 (Zurawski, et al., Immunol. Today 1994, 15:19-26). Experiments with transgenic and cytokine-deficient mice also implicate IL-13 as being important for IgE responses in vivo (Emson, et al, J. Exp Med., 1998, 188:399-404).

In addition to these immunostimulatory properties, IL-13 has important anti-inflammatory properties. IL-13 enhances the production of IL-1Rα and induces the release of the “decoy” IL-1RII. These molecules effectively bind IL-1 and therefore have anti-inflammatory activity. IL-13 also effectively downregulates the production of proinflammatory cytokines (e.g., IL-1α, IL-1β, IL-6, and TNF-α) and chemokines (e.g., IL-8, macrophage inflammatory protein-1α, and 1β and monocyte chemotactic protein-3).

More recently, IL-13 has been shown to play a critical role in mediating the parasite-induced immune and allergy response (Chiaramonte, et al., J. Immunol. 1999, 162:920-930). With respect to a role in asthma, IL-13 has been shown to be required for both airway hyper-responsiveness and mucus overproduction (Gavett, et al., Am J. Physiol. 1997, 272:L253-L261; Wills-Karp, et al., Science 1998, 282:2258-2261; Grunig, et al., Science 1998, 282:2261-2263). Further, Bousquet et al (N. Engl. J. Med. 1990, 323:1033-1039) demonstrated that the presence or absence of IL-13 in mouse airways correlated directly with the degree of airway eosinophilia, which is possibly the most vivid marker of allergic inflammation in asthma.

Cytokines elicit their responses by binding to specific high affinity cell-surface receptors on target cells and initiating a series of intracellular signal transduction pathways. The receptors of several cytokines and growth factors are homologous within their extracellular domains. These receptors have been grouped into families, the largest of which is the hematopoietin receptor superfamily which includes one or multiple chains of the receptors for erythropoietin, IL-2, IL-3, IL-4, IL-5, IL-6, IL-7, IL-9, IL-11, IL-12, IL-13, IL-15, GM-CSF and G-CSF.

Not only do IL-4 and IL-13 share significant structural homology, they are also closely linked through a common receptor signaling moiety, the IL-4 receptor alpha chain. The classical IL-4R complex, consisting of the IL-4Rα chain and the common γ-chain (γc), a shared component of the receptors for IL-2, IL-4, IL-7, IL-9, and IL-15, is specific for IL-4. On the other hand, the IL-4 Rα chain is also part of the IL-13R complex. IL-13R is expressed on B cells, monocytes, macrophages, basophils, eosinophils, mast cells, endothelial cells, keratinocytes, and certain tumor cells, such as renal cell carcinomas and glioblastomas. IL-I3R is usually present at 200 to 3000 sites per cell and binds IL-13 with high affinity (Kd 30 pmo/L). The high-affinity IL-13R complex consists of the 140-kd IL-4Rα chain, which binds IL-4 but not IL-13, and an IL-13 binding protein. Two different cDNAs encoding IL-13-binding proteins have been cloned recently and designated IL-13 Rα1 and IL-13 Rα2. The genes encoding these receptors are both mapped on the X chromosome; IL-13Rα1 consists of 427 amino acids and binds IL-13 with low affinity (Kd −4 nmol/L). IL-13Rα2 is a 380 amino acid protein, which binds IL-13 with high affinity (Kd −50 pmol/L) in the absence of the IL-4Rα chain. The human IL-13Rα1 and IL-13Rα2 chains are 27% homologous and are expressed as 65 to 70 kd glycosylated molecules.

IL-4R and IL-13R complexes share the signal transducing IL-4Rα chain. Therefore binding of IL-4 or IL-13 to IL-4R and IL-13R results in comparable signaling pathways. Both cytokines activate the JAK-1 and Tyk 2 kinases and induce tyrosine phosphorylation of the IL-4Rα chain. IL-13 binds to the IL-13 cell surface receptor complex (IL-13R). Phosphorylation of the IL-4Rα chain after binding of IL-13 to the IL-13R complex and IL-4 to the IL-4 and IL-13R complexes results in the recruitment, phosphorylation, and nuclear translocation of signal transducer and activator of transcription-6 and the activation of IL-13 and IL-4-responsive genes in various cell types expressing IL-13R and IL-4R complexes.

The IL-13Rα chain of the receptor is a membrane-bound protein and plays an important role in signaling of IL-13, as does the IL-R_{χ} chain, even though the _{χ} subunit does not bind IL-13. Two other proteins have been cloned and shown to play roles in IL-13R, namely the IL-13Rα1 and IL-13Rα2. Together, the IL-13Rα, IL-13Rα1, IL-13Rα2, and IL-13R_{χ} chains have been proposed to form four different types of IL-13R complexes, depending on the cell type and resulting in different IL-13 ligand binding properties for each combination. While the role of each subunit chain is unclear, Donaldson et al. (J. Immunol. 1998; 161:2317-2324) demonstrated that a synthetic, soluble IL-13Rα2/Fc fusion protein blocked the mitogenic response to IL-13 in mouse Ba/F3 cells. However, in spite of the recent advances, the precise role of IL-13 downstream effectors and pathways of IL-13 remain unclear.

Ischemia usually is a temporary shortage of oxygen in a part of the body. It can occur when an artery bringing blood to that part, such as the kidney, liver or heart, is narrowed by spasm or disease. Ischemic kidney or liver disease is marked by increased inflammation, breakdown of function, and can result in septic shock. Ischemic heart disease includes heart attack and related heart problems caused by narrowed coronary arteries and thus less blood and oxygen reaching the heart. As many as 3 to 4 million Americans may have ischemic episodes without knowing it. These people, who have ischemia without pain, have silent ischemia. They may have kidney failure or a heart attack with no prior warning. In addition, people with anemia, hypertension or angina (chest pain) also may have undiagnosed episodes of silent ischemia.

Silent ischemia often coexists with painful ischemia. Silent myocardial ischemia is clinically important because it is associated with poor prognosis after an event such as an episode of unstable angina or myocardial infarction. Most dramatically, it has been assumed to exist in patients in whom coronary artery disease presents itself as sudden cardiac death. Silent ischemia has also been found during exercise in survivors of cardiac arrest and in patients with life-threatening arrhythmias.

Acute kidney failure occurs when illness, infection, or injury damages the kidneys. Temporarily, the kidneys cannot adequately remove fluids and wastes from the body or maintain the proper level of certain kidney-regulated chemicals in the bloodstream. The kidneys are the body's natural filtration system. Waste products like urea and toxins, along with excess fluids, are removed from the bloodstream in the form of urine. Kidney (or renal) failure occurs when kidney functioning becomes impaired. Fluids and toxins begin to accumulate in the bloodstream, leading to inflammation, swelling, ischemia and eventually acute kidney failure and high blood pressure. Acute kidney failure appears most frequently as a complication of serious illness, like heart failure, liver failure, ischemia, dehydration, and excessive bleeding (hemorrhage). It may also be caused by an obstruction to the urinary tract or as a direct result of kidney disease, injury, or an adverse reaction to a medicine.

These conditions fall into three main categories: prerenal, postrenal, and intrarenal conditions. Prerenal conditions do not damage the kidney, but can cause diminished kidney function. They are the most common cause of acute renal failure, and include: dehydration, hemorrhage, septicemia (or sepsis), heart failure or liver failure. Postrenal conditions cause kidney failure by obstructing the urinary tract. These conditions include inflammation of the prostate gland in men (prostatitis), enlargement of the prostate gland (benign prostatic hypertrophy), bladder or pelvic tumors, and kidney stones (calculi). Intrarenal conditions involve kidney disease or direct injury to the kidneys. These conditions include: ischemia-reperfusion (lack of blood supply to the kidneys), acute inflammation of the glomeruli, or filters, of the kidney (glomerulonephritis) and Kidney infections (pyelitis or pyelonephritis).

Ischemia-reperfusion injury frequently occurs when the flow of blood to a region of the body is temporarily halted (ischemia) and then re-established (reperfusion). Ischemia-reperfusion injury can occur during certain surgical procedures, such as repair of certain aortic aneurysms and organ transplantation. Clinically ischemia-reperfusion injury is manifested by such complications as pulmonary dysfunction, including adult respiratory distress syndrome, renal dysfunction, consumptive coagulopathies including thrombocytopenia, fibrin deposition into the microvasculature and disseminated intravascular coagulopathy, transient and permanent spinal cord injury, cardiac arrhythmias and acute ischemic events, hepatic dysfunction including acute hepatocellular damage and necrosis, gastrointestinal dysfunction including hemorrhage and/or infarction and multisystem organ dysfunction (MSOD) or acute systemic inflammatory distress syndromes (SIRS). It is believed that ischemia-reperfusion injury is caused, at least in part, by the release of excess amounts of proinflammatory cytokines, such as TNF-alpha, IL-1, IL-6, and IL-8.

It has been reported that 459,841 deaths occurred in the United States in 1998 (one of every 5 deaths) due to cardiac ischemia-reperfusion. The estimated incidence of this disease is 1,100,000 new and recurrent cases of coronary attack per year, of which over 4 percent die. There are approximately 12,400,000 victims of angina (chest pain due to coronary heart disease), heart attack and other forms of coronary heart disease. From 1988 to 1998 the death rate from coronary heart disease declined 26.3 percent, but the actual number of deaths declined only 9.8 percent. Estimates are that 6,400,000 people in the United States suffer from ischemia related angina. An estimated 400,000 new cases of angina occur each year.

Current diagnostic procedures generally assess the extent of cardiac damage after clinical signs have appeared. At that point, however, the disease may have progressed to a critical extent, wherein irreparable damage may have already occurred. Furthermore, current diagnostic procedures for subjects suspected of having acute kidney failure involve blood and urine tests to determine the level of kidney function. A blood test will assess the levels of creatinine, blood urea nitrogen (BUN), uric acid, phosphate, sodium, and potassium. Urine samples will also be collected to assess protein loss and/or creatinine clearance.

Determining the cause of kidney failure is critical to proper treatment. A full assessment of the kidneys is necessary to determine if the underlying disease is treatable and if the kidney failure is chronic or acute. X rays, magnetic resonance imaging (MRI), computed tomography scan (CT), ultrasound, renal biopsy, and/or arteriogram of the kidneys are often used to determine the cause of kidney failure and level of remaining kidney function. X rays and ultrasound of the bladder and/or uterus may also be needed.

Because many of the illnesses and underlying conditions that often trigger acute kidney failure are critical, the prognosis for these patients many times is not good. Studies have estimated overall death rates for acute kidney failure at 42-88%. Many people, however, die because of the primary disease that has caused the kidney failure. Early recognition and prompt, appropriate treatment are key to patient recovery. Up to 10% of patients who experience acute kidney failure will suffer irreversible kidney damage. They will eventually go on to develop chronic kidney failure or end-stage renal disease. These patients will require long-term dialysis or kidney transplantation to replace their lost renal functioning and death may result.

What is currently needed therefore is a method for diagnosing and or treating ischemia related dysfunction. The present invention provides a novel gene, IAP (ischemia activated protein), which plays an important role in the inflammatory cytokine response and is an indicator of the onset of ischemia and reperfusion in such organs as the heart, kidney, and liver. The methods of the present invention are useful for the diagnosis and or early prevention of such maladies, including but not hereby limited to: pulmonary dysfunction, including adult respiratory distress syndrome, renal dysfunction, consumptive coagulopathies including thrombocytopenia, fibrin deposition into the microvasculature and disseminated intravascular coagulopathy, transient and permanent spinal cord injury, cardiac arrhythmias and acute ischemic events, hepatic dysfunction including acute hepatocellular damage and necrosis and other inflammatory kidney and liver diseases, gastrointestinal dysfunction including hemorrhage and/or infarction and multisystem organ dysfunction (MSOD) or acute systemic inflammatory distress syndromes (SIRS), septic shock, and the various inflammatory complications related to organ or tissue transplantation.

### SUMMARY OF THE INVENTION

In summary, cytokines are key modulators of inflammation that play a central role in the onset of ischemia and reperfusion. They participate in acute and chronic inflammation in a complex network of interactions. This invention is in part based on the observed result that an early event in the onset of ischemia is the upregulation of IAP mRNA in cells during ischemia at the initiation of the inflammatory response.

Hence, the present invention provides a novel gene, IAP, which encodes a soluble protein that is involved in the initiation of an inflammatory cytokine response and which may interact with the anti-inflammatory IL-13. The protein of the present invention may be involved as a central mediator in ischemia, reperfusion, asthma and other inflammation-induced pathological conditions. According to one aspect of this invention, an isolated polynucleotide for determining the onset of ischemia and/or increased cytokine activity is provided. The polynucleotide comprises a gene located on human chromosome 1.

Preferably, the polynucleotide comprises a sequence selected from the following group: SEQ ID NO:1; an allelic variant of SEQ ID NO:1; a sequence hybridizing with SEQ ID NO:1 or its complement under moderate hybridization and washing conditions; an antisense sequence to SEQ ID NO:1; a sequence encoding a polypeptide having an amino acid sequence of SEQ ID NO:2 with up to 30% conservative substitutions; SEQ ID NO:3; an allelic variant of SEQ ID NO:3; a sequence hybridizing with SEQ ID NO:3 or its complement under moderate hybridization and washing conditions; an antisense sequence to SEQ ID NO:3; SEQ ID NO:4; an allelic variant of SEQ ID NO:4; a sequence hybridizing with SEQ ID NO:4 or its complement under moderate hybridization and washing conditions; an antisense sequence to SEQ ID NO:4; a sequence encoding a polypeptide having an amino acid sequence of SEQ ID NO:5 with up to 30% conservative substitutions; SEQ ID NO:6; an allelic variant of SEQ ID NO:6; a sequence hybridizing with SEQ ID NO:6 or its complement under moderate hybridization and washing conditions; an antisense sequence to SEQ ID NO:6; a sequence encoding a polypeptide having an amino acid sequence of SEQ ID NO:7 with up to 30% conservative substitutions; SEQ ID NO:8; an allelic variant of SEQ ID NO:8; a sequence hybridizing with SEQ ID NO:8 or its complement under moderate hybridization and washing conditions; an antisense sequence to SEQ ID NO:8; and a sequence encoding a polypeptide having an amino acid sequence of SEQ ID NO:9 with up to 30% conservative substitutions.

Another aspect of the invention features a recombinant DNA molecule comprising a vector having an insert that includes part or all of an IAP, or its antisense, polynucleotide and cells transformed with the recombinant DNA molecule. Preferably, the cells are murine, human, bovine, or rat cells. Most preferably, the cells are kidney, liver, heart or brain cells from one of the aforementioned organisms.

The invention also features an isolated polypeptide produced by expression of the IAP polynucleotides described above. Antibodies immunologically specific for the protein, or one or more epitopes thereof, are also provided.

In another aspect, the invention relates to methods for using such polypeptides and polynucleotides, for detecting the early onset of chronic asthma, psoriasis, stroke, ischemia, reperfusion, leishmaniasis, helminthiasis, hypoxia, or other causes of renal, liver or heart failure in a mammal, where increased cytokine activity is known to play a role. The present invention is implicated in diseases and conditions such as renal failure, septic shock, stroke, ischemia and reperfusion, among others. In a more particular embodiment, the invention relates to methods and compositions for detecting, in particular, ischemia, specifically silent ischemia, by obtaining a test sample from a mammal, measuring the level of IAP mRNA or protein in the test sample, and determining if the level of IAP mRNA or protein measured in the test sample correlates with elevated levels indicative of an ischemia associated disease.

The methods and compositions of the present invention allow for early detection of ischemia, reperfusion or hypoxia, i.e., conditions that lead to renal or liver failure, angina and other forms of heart failure. Using the instant methods, once ischemia, reperfusion or hypoxia has been detected, the patient can present himself or herself to an emergency medical facility, where measures can be taken in order to prevent renal, liver or heart failure from occurring. These measures include, but are not limited to, blood transfusion, medication, dialysis, hemofiltration, angioplasty, coronary bypass surgery, or administration of one or more anticoagulant or thrombolytic drugs.

The purpose of such preventive measures is to alleviate the ischemic or hypoxic conditions prior to the onset of organ failure. In contrast, today the above measures are used after an ischemic condition has occurred, or while the patient is experiencing organ failure. The present invention, however, allows for early detection of conditions that cause organ failure, prior to the onset of the symptoms and the tissue damage associated therewith.

The methods and compositions of the present invention allow physicians and other health care professionals to determine the success of a preventative procedure immediately following the procedure. For instance, following angioplasty, stent placement in a cardiac artery, or organ transplantation, the level of IAP mRNA or protein can be monitored as described herein to determine whether the ischemic, reperfusion or hypoxic conditions are being alleviated. Thus, the success of the operation can be immediately determined. If the procedure did not result in the desired results, as determined by the level of the IAP mRNA or protein, then further procedures can be employed prior to the patient suffering a complete or partial organ failure.

### BRIEF DESCRIPTION OF THE DRAWING

The invention is best understood from the following detailed description when read in connection with the accompanying drawings, in which:

FIG. 1 is a schematic representation of targeted differential display (TDD).

FIG. 2 is a Northern Blot showing mRNA Expression of IAP mRNA in various mouse tissues.

FIG. 3 is a Northern Blot showing activation of the IAP gene by ischemia-reperfusion in mouse kidney.

FIG. 4 is polyacrylamide gel electrophoresis showing expression of mouse IAP protein in E. Coli cells.

FIG. 5a is a Northern Blot again showing mRNA Expression of IAP mRNA in various mouse tissues.

FIG. 5b is a Northern Blot again showing activation of the IAP gene by ischemia/reperfusion in mouse kidney.

FIG. 6 is a graph showing the measurement of IAP mRNA activation during kidney ischemia-reperfusion.

FIG. 7 is a Western Blot showing the overexpression of IAP in Hela cells.

### DETAILED DESCRIPTION OF THE INVENTION

Definitions

Various terms relating to the biological molecules of the present invention are used throughout the specification and claims.

“IAP” refers generally to an IAP soluble polypeptide that is highly inducible in ischemia at time points when an inflammatory response starts, in accordance with the present invention, which is described in detail herein above and throughout the specification.

“IAP activity or IAP polypeptide activity” or “biological activity of the IAP or IAP polypeptide” refers to the metabolic or physiologic function of said IAP including similar activities or improved activities or these activities with decreased undesirable side-effects. Also included are antigenic and immunogenic activities of said IAP. In particular, IAP encodes a soluble protein that has homology at its C-terminal and may bind to IL-13.

“IAP gene” refers to a polynucleotide as defined above in accordance with the present invention, which encodes an IAP polypeptide.

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated”, as the term is employed herein.

“Polynucleotide” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA.

The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as posttranslational processing, or by chemical modification techniques that are well known in the art. Such modifications are well described in basic texts and in more detailed monographs; as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslation natural processes or may be made by synthetic methods.

Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, Proteins—Structure And Molecular Properties, 2nd Ed., T. E. Creighton, W. H. Freeman and Company, New York, 1993 and Wold, F., “Posttranslational Protein Modifications: Perspectives and Prospects, pgs. 1-12 in “Posttranslational Covalent Modification Of Proteins”, B, C. Johnson, Ed., Academic Press, New York, 1983; Seifter et al., “Analysis for protein modifications and nonprotein cofactors”, Meth Enzymol (1990) 182:626-646 and Rattan et al, “Protein Synthesis: Posttranslational Modifications and Aging”, Ann AIY Acad Sci (1992) 663:48-62.

“Variant” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical.

A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

The term “substantially the same” refers to nucleic acid or amino acid sequences having sequence variation that do not materially affect the nature of the protein (i.e. the structure, stability characteristics, substrate specificity and/or biological activity of the protein). With particular reference to nucleic acid sequences, the term “substantially the same” is intended to refer to the coding region and to conserved sequences governing expression, and refers primarily to degenerate codons encoding the same amino acid, or alternate codons encoding conservative substitute amino acids in the encoded polypeptide. With reference to amino acid sequences, the term “substantially the same” refers generally to conservative substitutions and/or variations in regions of the polypeptide not involved in determination of structure or function.

The terms “percent identical” and “percent similar” are also used herein in comparisons among amino acid and nucleic acid sequences. When referring to amino acid sequences, “identity” or “percent identical” refers to the percent of the amino acids of the subject amino acid sequence that have been matched to identical amino acids in the compared amino acid sequence by a sequence analysis program. “Percent similar” refers to the percent of the amino acids of the subject amino acid sequence that have been matched to identical or conserved amino acids. Conserved amino acids are those which differ in structure but are similar in physical properties such that the exchange of one for another would not appreciably change the tertiary structure of the resulting protein. Conservative substitutions are defined in Taylor (1986, J. Theor. Biol. H 9:205). When referring to nucleic acid-molecules, “percent identical” refers to the percent of the nucleotides of the subject nucleic acid sequence that have been matched to identical nucleotides by a sequence analysis program.

“Identity” and “similarity” can be readily calculated by known methods. Nucleic acid sequences and amino acid sequences can be compared using computer programs that align the similar sequences of the nucleic or amino acids thus define the differences. In preferred methodologies, the-BLAST programs (NCBI) and parameters used therein are employed, and the DNAstar system (Madison, Wis.) is used to align sequence fragments of genomic DNA sequences. However, equivalent alignments and similarity/identity assessments can be obtained through the use of any standard alignment software. For instance, the GCG Wisconsin Package version 9.1, available from the Genetics Computer Group in Madison, Wis., and the default parameters used (gap creation penalty=12, gap extension penalty=4) by that program may also be used to compare sequence identity and similarity.

With respect to single-stranded nucleic acid molecules, the term “specifically hybridizing” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule, to the substantial exclusion of hybridization of the oligonucleotide with single-stranded nucleic acids of non-complementary sequence.

With respect to oligonucleotides, but not limited thereto, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “substantially pure” refers to a “preparation comprising at least 50-60% by weight the compound of interest (e.g., nucleic acid, oligonucleotide, protein, etc.). More preferably, the preparation comprises at least 75% by weight, and most preferably 90-99% by weight, the compound of interest. Purity is measured by methods appropriate to the compound of interest (e.g. chromatographic methods, agarose or polyacrylamide gel electrophoresis, HPLC analysis, and the like).

A “coding sequence” or “coding region” refers to a nucleic acid molecule having sequence information necessary to produce a gene product, when the sequence is expressed.

The term “operably linked” or “operably inserted” means that the regulatory sequences necessary for expression of the coding sequence are placed in a nucleic acid molecule in the appropriate positions relative to the coding sequence so as to enable expression of the coding sequence. This same definition is sometimes applied to the arrangement other transcription control elements (e.g. enhancers) in an expression vector.

Transcriptional and translational control sequences are DNA regulatory sequences, such as promoters, enhancers, polyadenylation signals, terminators, and the like, that provide for the expression of a coding sequence in a host cell.

The terms “promoter”, “promoter region” or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

A “vector” is a replicon, such as plasmid, phage, cosmid, or virus to which another nucleic acid segment may be operably inserted so as to bring about the replication or expression of the segment.

The term “nucleic acid construct” or “DNA construct” is sometimes used to refer to a coding sequence or sequences operably linked to appropriate regulatory sequences and inserted into a vector for transforming a cell. This term may be used interchangeably with the term “transforming DNA”. Such a nucleic acid construct may contain a coding sequence for a gene product of interest, along with a selectable marker gene and/or a reporter gene.

The term “selectable marker gene” refers to a gene encoding a product that, when expressed, confers a selectable phenotype such as antibiotic resistance on a transformed cell.

The term “reporter gene” refers to a gene that encodes a product that is detectable by standard methods, either directly or indirectly.

A “heterologous” region of a nucleic acid construct is an identifiable segment (or segments) of the nucleic acid molecule within a larger molecule that is not found in association with the larger molecule in nature. Thus, when the heterologous region encodes a mammalian gene, the gene will usually be flanked by DNA that does not flank the mammalian genomic DNA in the genome of the source organism. In another example, a heterologous region is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

The term “DNA construct”, as defined above, is also used to refer to a heterologous region, particularly one constructed for use in transformation of a cell. A cell has been “transformed” or “transfected” by exogenous or heterologous DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA.

A “clone” is a population of cells derived from a single cell or common ancestor by mitosis.

A “cell line” is a clone of a primary cell that is capable of stable growth in vitro for many generations.

“Ischemia” means a condition where an organ receives insufficient blood supply, whereas “hypoxia” means a condition where the organ receives insufficient oxygen.

The term “mammal” refers to such organisms as mice, rats, rabbits, goats, horse, sheep, cattle, cats, dogs, pigs, more preferably monkeys and apes, and most preferably humans.

In preferred embodiments, the measuring step of the methods of the invention comprises measuring the marker level by a method selected from the group consisting of chromatography, immunoassay, enzymatic assay, and spectroscopy, where the cardiac marker is directly or indirectly detected. “Marker level” means the amount of the marker in the sample or in the mammal, and refers to units of concentration, mass, moles, volume, preferably concentration, or other measure indicating the amount of marker present in the sample.

In a “quantitative” measurement, the step of measuring results in the production of a value that accurately shows the level of the cardiac marker in the test sample. In a “semi-quantitative” measurement, the step of measuring results in the indication of whether the level of the cardiac marker is within a particular range. Semi-quantitative methods include, for example, but are not limited to, color indicators or depiction of certain symbols, where each color or symbol represents a concentration range.

“Antibodies” as used herein includes polyclonal and monoclonal antibodies, chimeric, single chain, and humanized antibodies, as well as Fab fragments, including the products of a Fab or other immunoglobulin expression library. With respect to antibodies, the term, “immunologically specific” refers to antibodies that bind to one or more epitopes of a protein of interest, but which do not substantially recognize and bind other molecules in a sample containing a mixed population of antigenic biological molecules.

The term “specific binding affinity” is meant that the antibody or antibody fragment binds to target compounds with greater affinity than it binds to other compounds under specified conditions. Antibodies or antibody fragments having specific binding affinity to a compound may be used in methods for detecting the presence and/or amount of the compound in a sample by contacting the sample with the antibody or antibody fragment under conditions such that an immunocomplex forms and detecting the presence and/or amount of the compound conjugated to the antibody or antibody fragment.

The term “polyclonal” refers to antibodies that are heterogeneous populations of antibody molecules derived from the sera of animals immunized with an antigen or an antigenic functional derivative thereof. For the production of polyclonal antibodies, various host animals may be immunized by injection with the antigen. Various adjuvants may be used to increase the immunological response, depending on the host species.

“Monoclonal antibodies” are substantially homogenous populations of antibodies to a particular antigen. They may be obtained by any technique that provides for the production of antibody molecules by continuous cell lines in culture. Monoclonal antibodies may be obtained by methods known to those skilled in the art. See, for example, Kohler, et al., Nature 256:495-497, 1975, and U.S. Pat. No. 4,376,110.

The term “antibody fragment” refers to a portion of an antibody, often the hypervariable region and portions of the surrounding heavy and light chains, that displays specific binding affinity for a particular molecule. A hypervariable region is a portion of an antibody that physically binds to the target compound. The term “antibody fragment” also includes single charge antibodies.

Polynucleotides

The present invention provides a novel gene, IAP, which is involved in the inflammatory response and other interrelated physiological processes of the cytokine-mediated immune response. The IAP gene and protein of the present invention share a portion of sequence homology to IL-13Rα chain of the membrane IL-13SR receptor complex. However, unlike the IL-13Rα chain, IAP is not a membrane-bound protein, but rather is soluble in form. The summary of the invention described above is non-limiting and other features and advantages the invention will be apparent from the-following detailed description, and from the claims.

The present invention concerns methods and compositions for early diagnosis of ischemic renal or liver dysfunction as well as heart or other forms of heart failure by detecting levels of IAP mRNA or protein, and/or its metabolites, alone or in conjunction with one or more other markers in a test sample from a mammal. The invention is based on the inventor's discovery that an early event in the course of organ failure, for example that caused by ischemia, is excess production of IAP mRNA and/or protein.

This invention is based in part on the discovery that in ischemic subjects, the levels of IAP mRNA and/or protein are significantly higher than those detected in non-ischemic controls. Based on the results obtained, levels of IAP mRNA or protein are diagnostic of organ failure associated with ischemia, reperfusion or hypoxia.

Immunodiagnostic assays, using a variety of known methods, can also be used to detect IAP mRNA or protein, in body cells and fluids, including blood or serum. Antibodies and antibody fragments specific for IAP mRNA or protein can be produced and used to quantitatively or semiquantitatively detect the presence of IAP mRNA or protein in cell extracts, whole blood, serum, urine or other body fluids using standard immunoassays. Similarly, immunoassays that detect the presence of IAP antibodies in body fluids can be used to indirectly test for increased levels of such marker(s) in patients with chronic conditions associated with heart failure, including chronic ischemia, reperfusion and hypoxia.

As described in detail in Example 1, the IAP gene was first identified and cloned from mouse and human. The human IAP gene exists in a spliced form, SEQ ID NO:1 and an unspliced form, SEQ ID NO:3. Computer analysis of the protein sequence of the murine sequence using PredictProtein software identified the murine sequence as haring homology to the extracellular domain of the IL-13 alpha chain receptor. Computer analysis (BLAST search) of the non-redundant databases revealed the presence of the highly homologous sequences in EST databases from bovine and rat libraries, SEQ ID NO:6 and SEQ ID NO:8, respectively.

The IAP polynucleotides of the present invention include isolated polynucleotides encoding the IAP polypeptides and fragments, and polynucleotides closely related thereto. More specifically, IAP polynucleotides of the invention include a polynucleotide comprising the human nucleotide sequences contained in SEQ ID NO:1 or SEQ ID NO:3 encoding a IAP polypeptide of SEQ ID NO:2, and polynucleotides having the particular sequence of SEQ ID NO:1 or SEQ ID NO:3. Also included are IAP polynucleotides comprising the murine sequence of SEQ ID NO:4 encoding a IAP polypeptide of SEQ ID NO:5, and polynucleotides having the particular sequence of SEQ ID NO:4. Still further included are the IAP polynucleotides comprising the bovine sequence of SEQ ID NO:6 encoding a IAP polypeptide of SEQ ID NO:7, and polynucleotides having the particular sequence of SEQ ID NO:6. Even still further included are the IAP polynucleotides comprising the rat sequence of SEQ ID NO:8 encoding the polypeptide of SEQ ID NO:9, and polynucleotides having the particular sequence of SEQ ID NO:8.

IAP polynucleotides further include a polynucleotide comprising a nucleotide sequence that has at least 70% identity over its entire length to a nucleotide sequence encoding the IAP polypeptide of SEQ ID NO:2, SEQ ID NO:5, SEQ ID NO:7, or SEQ ID NO:9, and a polynucleotide comprising a nucleotide sequence that is at least 70% identical to that of SEQ ID NO:1, SEQ ID NO:3, SEQ ID NO:4, SEQ ID NO:6, or SEQ ID NO:8, over its entire length. In this regard, polynucleotides with at least 70% are preferred, more preferably at least 80% even more preferably at least 90% identity, yet more preferably at least 95% identity, 97% are highly preferred and those with at least 98-99% are most highly preferred, with at least 99% being the most preferred. Also included under IAP polynucleotides are a nucleotide sequence which has sufficient identity to a nucleotide sequence contained in SEQ ID NO:1, SEQ ID NO:3, SEQ ID NO:4, SEQ ID NO:6 or SEQ ID NO:8 to hybridize under conditions useable for amplification or for use as a probe or marker. The invention also provides polynucleotides that are complementary to such IAP polynucleotides.

Also included in the present invention are polynucleotides encoding polypeptides which have at least 70% identity, preferably at least 80% identity, more preferably at least 90% identity, yet more preferably at least 95% identity, even more preferably at least 97-99% identity, to the amino acid sequence of SEQ ID NO:2, SEQ ID NO:5, SEQ ID NO:7 or SEQ ID NO:9 over the entire length of the recited amino acid sequences.

The nucleotide sequences encoding the IAP polypeptide of SEQ ID NO:2 may be identical to the polypeptide encoding sequence contained in SEQ ID NO:1 or SEQ ID NO:3, or it may be a sequence, which as a result of the redundancy (degeneracy) of the genetic code, also encodes the polypeptide of SEQ ID NO:2. Similarly, the nucleotide sequences encoding the polypeptides of SEQ ID NO:5, SEQ ID NO:7, or SEQ ID NO:9 may be identical to the polypeptide encoding sequences of SEQ ID NO:4, SEQ ID NO:6, or SEQ ID NO:8, or they may be a sequence, which as a result of the redundancy (degeneracy) of the genetic code, also encode the polypeptides of SEQ ID NO:5, SEQ ID NO:7, or SEQ ID NO:9.

When the polynucleotides of the invention are used for the recombinant production of IAP polypeptide, the polynucleotide may include the coding sequence for the mature polypeptide or a fragment thereof, by itself; the coding sequence for the mature polypeptide or fragment in reading frame with other coding sequences, such as those encoding a leader or secretory sequence, a pre-, or pro- or prepro-protein sequence, or other fusion peptide portions. For example, a marker sequence which facilitates purification of the fused polypeptide can be encoded. The polynucleotide may also contain non-coding 5′ and 3′ sequences, such as transcribed, non translated sequences, splicing and polyadenylation signals, ribosome binding sites and sequences that stabilize mRNA.

Thus, this invention provides oligonucleotides (sense or antisense strands of DNA or RNA) having sequences capable of hybridizing with at least one sequence of a nucleic acid molecule encoding the protein of the present invention. Such oligonucleotides are useful as probes for detecting IAP genes or transcripts. In one preferred embodiment, oligonucleotides for use as probes or primers are based on rationally-selected amino acid sequences chosen from SEQ ID NO:1, SEQ ID NO:3, SEQ ID NO:4, SEQ ID NO:6 or SEQ ID NO:8. In preferred embodiments, the amino acid sequence information is used to make degenerate oligonucleotide sequences as is commonly done by those skilled in the art. In other preferred embodiments the degenerate oligonucleotides are used to screen cDNA libraries from human, mouse, bovine and rat.

IAP polynucleotides of the present invention may be prepared by two general methods: (1) they may be synthesized from appropriate nucleotide triphosphates, or (2) they may be isolated from biological sources. Both methods utilize protocols well known in the art. The availability of nucleotide sequence information, such as the cDNA having SEQ ID NO:1 or SEQ ID NO:3, enables preparation of an isolated nucleic acid molecule of the invention by oligonucleotide synthesis.

Synthetic oligonucleotides may be prepared by the phosphoramadite method employed in the Applied Biosystems 38A DNA Synthesizer or similar devices. The resultant construct may be purified according to methods known in the art, such as high performance liquid chromatography (HPLC). Long, double-stranded polynucleotides, must be synthesized in stages, due to the size limitations inherent in current oligonucleotide synthetic methods. Thus, for example, a long double-stranded molecule may be synthesized as several smaller segments of appropriate complementarity. Complementary segments thus produced may be annealed such that each segment possesses appropriate cohesive termini for attachment of an adjacent segment. Adjacent segments may be ligated by annealing cohesive termini in the presence of DNA ligase to construct an entire long double-stranded molecule. A synthetic DNA molecule so constructed may then be cloned and amplified in an appropriate vector.

IAP genes also may be isolated from appropriate biological sources using methods known in the art. In the exemplary embodiment of the invention, IAP may be isolated from genomic libraries of human, mouse, bovine or rat. In alternative embodiments, cDNA clones of IAP may be isolated, such as what has been isolated from human, murine, bovine and rat cDNA libraries. A preferred means for isolating IAP genes is PCR amplification using genomic or cDNA templates and IAP specific primers. Genomic and cDNA libraries are commercially available, and can also be made by procedures well known in the art. In positions of degeneracy where more than one nucleic acid residue could be used to encode the appropriate amino acid residue, all the appropriate nucleic acid residues may be incorporated to create a mixed oligonucleotide population, or a neutral base such as inosine may be used. The strategy of oligonucleotide design is well known in the art.

Alternatively, PCR primers may be designed by the above method to match the coding sequences of a human, murine, bovine, or rat protein and these primers used to amplify the native nucleic acids from isolated cDNA or genomic DNA.

In accordance with the present invention, nucleic acids having the appropriate level sequence homology (i.e., 70% identity or greater) with part or all the coding regions of SEQ ID NO:1, SEQ ID NO:3, SEQ ID NO:4, SEQ ID NO:6 or SEQ ID NO:8 may be identified by using hybridization and washing conditions of appropriate stringency. For example, hybridizations may be performed, according to the method of Sambrook et al., using a hybridization solution comprising: 1.0% SDS, up to 50% formamide, 5×SSC (150 mM NaCl, 15 mM trisodium citrate), 0.05% sodium pyrophosphate (pH7.6), 5×Denhardt's solution, and 100 microgram/ml denatured, sheared salmon sperm DNA. Hybridization is carried out at 37-42° C. for at least six hours. Following hybridization, filters are washed as follows: (1) 5 minutes at room temperature in 2×SSC and 1% SDS; (2) 15 minutes at room temperature in 2×SSC and 0.1% SDS; (3) 30 minutes to 1 hour at 37° C. in 2×SSC and 0.1% SDS; (4) 2 hours at 45-55° C. in 2×SSC and 0.1% SDS, changing the solution every 30 minutes.

One common formula for calculating the stringency conditions required to achieve hybridization between nucleic acid molecules of a specified percent identity is set forth by (Sambrook et al., 1989, supra):

Tₘ=81.5° C.+16.6Log [Na+]+0.4 1 (% G-C)−0.63 (% formamide)−600/#bp in duplex

As an illustration of the above formula, using [N+]=[0.368]0 and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and wash in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and wash in 1×SSC and 0.5% SDS at 6-5° C. for 15 minutes. A very high stringency hybridization is defined as hybridization in 6×SSC, S×Denhardt's solution, 0.5% SDS and 100 μg/ml denatured salmon sperm DNA at 42° C., and wash in 0.1×SSC and 0.5% SDS at 65° C. for 15 minutes.

Nucleic acids of the present invention may be maintained as DNA in any convenient cloning vector. In a preferred embodiment, clones are maintained in plasmid cloning/expression vector, such as pBluescript (Stratagene, La Jolla, Calif.), which is propagated in a suitable E. coli host cell.

The IAP poly-nucleotides may be used for a variety of purposes in accordance with the present invention. DNA, RNA, or fragments thereof may be used as probes to detect the presence of and/or expression of IAP genes. Methods in which IAP nucleic acids may be utilized as probes for such assays include, but are not limited to: (1) in situ hybridization; (2) Southern hybridization (3) Northern hybridization; and (4) assorted amplification reactions such as polymerase chain reaction (PCR).

The IAP nucleic acids may also be utilized as probes to identify related genes from other species. As is well known in the art, hybridization stringencies may be adjusted to allow hybridization of nucleic acid probes with complementary sequences of varying degrees of homology.

As described above, IAP nucleic acids may be used to produce large quantities of substantially pure IAP proteins, or selected portions thereof.

The IAP nucleic acids of the present invention can be used to identify and isolate other members of the inflammatory pathway(s) in which IAP is involved. A yeast two-hybrid system can be used to identify proteins that physically interact with the IAP protein, as well as isolate their nucleic acids. In this system, the coding sequence of the protein of interest is operably linked to the coding sequence of half of an activator protein. This construct is used to transform a yeast cell library that has been transformed with DNA constructs that contain the coding sequence for the other half of the activator protein operably linked to a random coding sequence from the organism of interest. When the protein made by the random coding sequence from the library interacts with the protein of interest, the two halves of the activator protein are physically associated and form a functional unit that activates the reporter gene.

In accordance with the present invention, all or part of the human, mouse, bovine or rat IAP coding sequence may be operably linked to the coding sequence of the first half of the activator, and the library of random coding sequences may be constructed with cDNA from human, mouse, bovine or rat and operably linked to the coding sequence of the second half of the activator protein. Several activator protein/reporter genes are customarily used in the yeast two hybrid system, the Gal4/LacZ system (see Clark et al., 1998 PNAS 95:5401-5406), among others.

The nucleotide sequences of the present invention are also valuable for chromosome localization. The sequence is specifically targeted to, and can hybridize with, a particular location on an individual human chromosome. The mapping of relevant sequences to chromosomes according to the present invention is an important first step in correlating those sequences with gene associated disease. Once a sequence has been mapped to a precise chromosomal location, the physical position of the sequence on the chromosome can be correlated with genetic map data. The relationship between genes and diseases that have been mapped to the same chromosomal region are then identified through linkage analysis (co-inheritance of physically adjacent genes). The human genes of the present invention including SEQ ID NOS:1 and 3 map to human chromosome 1, in the Hs. 64679 cluster (DIS2843 -DIS417; 48.8-81.6 cM; Physical position—111.87 cR3000:P0.64).

Polypeptides

In one aspect, the present invention relates to human IAP polypeptides (or IAP proteins). The human IAP polypeptides include the polypeptide of SEQ ID NO:2; as well as polypeptides comprising the amino acid sequence of SEQ ID NO:2; and polypeptides comprising the amino acid sequence which have at least 70% identity to that of SEQ ID NO:2, over its entire length. Preferably IAP polypeptide exhibit at least one biological activity of IAP. The present invention further provides for a polypeptide which comprises an amino acid sequence which has at least 80% identity, more preferably at least 90% identity, yet more preferably at least 95% identity, most preferably at least 97-99% identity, to that of SEQ ID NO:2 over the entire length of SEQ ID NO:2.

In another aspect, the present invention relates to mouse IAP polypeptides (or IAP proteins). The murine IAP polypeptides include the polypeptide of SEQ ID NO:5; as well as polypeptides comprising the amino acid sequence of SEQ ID NO:5; and polypeptides comprising the amino acid sequence which have at least 70% identity to that of SEQ ID NO:5, over its entire length. Preferably the mouse IAP polypeptides exhibit at least one biological activity of IAP. The present invention further provides for a polypeptide which comprises an amino acid sequence which has at least 80% identity, more preferably at least 90% identity, yet more preferably at least 95% identity, most preferably at least 97-99% identity, to that of SEQ ID NO:5 over the entire length of SEQ ID NO:5.

In another aspect, the present invention relates to bovine IAP polypeptides (or IAP proteins). The bovine IAP polypeptides include the polypeptide of SEQ ID NO:7; as well as polypeptides comprising the amino acid sequence of SEQ ID NO:7; and polypeptides comprising the amino acid sequence which have at least 70% identity to that of SEQ ID NO:7, over its entire length. Preferably the bovine IAP polypeptides exhibit at least one biological activity of IAP. The present invention further provides for a polypeptide which comprises an amino acid sequence which has at least 80% identity, more preferably at least 90% identity, yet more preferably at least 95% identity, most preferably at least 97-99% identity, to that of SEQ ID NO:7 over the entire length of SEQ ID NO:7.

In still another aspect, the present invention relates to rat IAP polypeptides (or IAP proteins). The rat IAP polypeptides include the polypeptide of SEQ ID NO:9; as well as polypeptides comprising the amino acid sequence of SEQ ID NO:9; and polypeptides comprising the amino acid sequence which have at least 70% identity to that of SEQ ID NO:9, over its entire length. Preferably the rat IAP polypeptides exhibit at least one biological activity of IAP. The present invention further provides for a polypeptide which comprises an amino acid sequence which has at least 80% identity, more preferably at least 90% identity, yet more preferably at least 95% identity, most preferably at least 97-99% identity, to that of SEQ ID NO:9 over the entire length of SEQ ID NO:9.

The IAP polypeptides may be in the form of the “mature” protein or may be a part of a larger protein such as a fusion protein. It is often advantageous to include an additional amino acid sequence which contains secretory or leader sequences, pro-sequences, sequences which aid in purification such as multiple histidine residues, or an additional sequence for stability during recombinant production.

Fragments of the IAP polypeptides are also included in the invention. A fragment is a polypeptide having an amino acid sequence that entirely is the same as part, but not all, of the amino acid sequence of the aforementioned IAP polypeptides. Preferred fragments include, for example, truncation polypeptides having the amino acid sequence of IAP polypeptides, except for deletion of a continuous series of residues that includes the amino terminus, or a continuous series of residues that includes the carboxyl terminus or deletion of two continuous series of residues, one including the amino terminus and one including the carboxyl terminus. Also preferred are fragments characterized by structural or functional attributes such as fragments that comprise alpha-helix and alpha-helix forming regions, beta-sheet and beta-sheet forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Other preferred fragments are biologically active fragments. Biologically active fragments are those that mediate IAP activity, including those with a similar activity or an improved activity, or with a decreased undesirable activity. Also included are those that are antigenic or immunogenic in an animal, especially in a human.

Preferably, all of these polypeptide fragments retain the biological activity of the IAP, including antigenic activity. Variants of the defined sequence and fragments also form part of the present invention. Preferred variants are those that vary from the referents by conservative amino acid substitutions.

The IAP polypeptides of the invention can be prepared in any suitable manner. If produced in situ, the polypeptides may be purified from appropriate sources, e.g., cells from human, mouse, bovine or rat.

Alternatively, the availability of nucleic acid molecules encoding the polypeptides enables production of the proteins using in vitro expression methods known in the art. For example, a cDNA or gene may be cloned into an appropriate in vitro transcription vector, for in vitro transcription, followed by cell-free translation in a suitable cell-free translation system. In vitro transcription and translation systems are commercially available, e.g., from Promega Biotech, Madison, Wis., or BRL, Rockville, Md. While in vitro transcription and translation is not the method of choice for preparing large quantities of the protein, it is ideal for preparing small amounts of native or mutant proteins for research purposes, particularly since it allows the incorporation of radioactive nucleotides.

According to a preferred embodiment, larger quantities of IAP encoded polypeptide may be produced by expression in a suitable procaryotic or eukaryotic system. For example, part or all of a DNA molecule, such as the coding portion of SEQ ID NO:1, SEQ ID NO:3, SEQ ID NO:4, SEQ ID NO:6, or SEQ ID NO:8, may be inserted into a plasmid vector adapted for expression in a bacterial cell (such as E. coli) or a yeast cell (such as Saccharomyces cerevisiae), or into a baculovirus vector for expression in an insect cell. Such vectors comprise the regulatory elements necessary for expression of the DNA in the host cell, positioned in such a manner as to permit expression of the DNA into the host cell. Such regulatory elements required for expression include promoter sequences, transcription initiation sequences and, optionally, enhancer sequences.

Secretion signals may be used to facilitate purification of the resulting protein. The coding sequence for the secretion peptide is operably linked to the 5′ end of the coding sequence for the protein, and this hybrid nucleic acid molecule is inserted into a plasmid adapted to express the protein in the host cell of choice. Plasmids specifically designed to express and secrete foreign proteins are available from commercial sources. For example, if expression and secretion is desired in E. coli, commonly used plasmids include pTrcPPA (Pharmacia); pPROK-C and pKK233-2 (Clontech); and pNH8a, pNH16a, pcDNAII and pAX (Stratagene), among others.

The IAP proteins produced by in vitro transcription and translation or by gene expression in a recombinant procaryotic or eukaryotic system may be purified according to methods known in the art. Recombinant proteins can be purified by affinity separation, such as by immunological interaction with antibodies that bind specifically to the recombinant protein or fusion proteins such as His tags, as described below. Such methods are commonly used by skilled practitioners.

As mentioned, the proteins can be produced and fused to a “tag” protein in order to facilitate subsequent purification. These fusion proteins are produced by operably-linking the nucleic acid coding sequence of the “tag” protein to the coding sequence of the protein of interest, and expressing the fused protein by standard methods. Systems are commercially available that comprise a plasmid containing an expression cassette with the “tag” protein coding sequence and a polylinker into which a coding sequence of interest can be operably ligated. These fusion protein systems further provide chromatography matrices or beads that specifically bind the “tag” protein thereby facilitating the fusion protein purification. These fusion protein systems often have the recognition sequence of a protease at or near the junction of the “tag” protein and the protein of interest so that the “tag” protein can be removed if desired. Fusion protein systems include, but are not limited to, the His-6-tag system (Quiagen) and the glutathione-S-transferase system (Phannacia).

The IAP proteins of the invention, prepared by one of the aforementioned methods, may be analyzed according to standard procedures. For example, the protein may be subjected to amino acid composition, amino acid sequence, or protein concentration analysis according to known methods.

Using appropriate amino acid sequence information, synthetic IAP proteins of the present invention may be prepared by various synthetic methods of peptide synthesis via condensation of one or more amino acid residues, in accordance with conventional peptide synthesis methods. Preferably, peptides are synthesized according to standard solid-phase methodologies, such as may be performed on an Applied Biosystems Model 430A peptide synthesizer (Applied Blosystems, Foster City, Calif.), according to manufacturer's instructions. Other methods of synthesizing peptides or peptidomimetics, either by solid phase methodologies or in liquid phase, are well known to those skilled in the art.

The IAP protein can be used as a label in many in vitro applications currently used. Purified IAP can be covalently linked to other proteins by methods well known in the art, and used as a marker protein. The purified IAP protein can be covalently linked to a protein of interest in order to determine localization. In particularly preferred embodiments, a linker of 4 to 20 amino acids is used to separate IAP from the desired protein. This application may be used in living cells by micro-injecting the linked proteins. The IAP may also be linked to antibodies and used thus for localization in fixed and sectioned cells. The IAP may be linked to purified cellular proteins and used to identify binding proteins and nucleic acids in assays in vitro, using methods well known in the art.

The IAP protein can also be linked to nucleic acids and used to advantage. Applications for nucleic acid-linked IAP include, but are not limited, to FISH (fluorescent in situ hybridization), and labeling probes in standard methods utilizing nucleic acid hybridization.

The IAP proteins of the present invention can be used to identify binding partners of IAP. In these assays, the first protein of interest is allowed to form a physical interaction with the unknown binding protein(s), often in a heterologous solution of proteins. The complex of proteins is then isolated, and the nature of the protein complex is determined. This procedure is greatly facilitated by a simple method for isolating the IAP protein. For example, immunologically-specific antibodies can be used to precipitate the IAP protein, or the IAP protein can be bound to beads that can be easily purified. Such beads can be magnetized, or simply dense enough to be separated form the non-associated protein by centrifugation.

In another aspect, the invention relates to methods for using such polypeptides and polynucleotides, for detecting the early onset of chronic asthma, psoriasis, renal, liver or other organ failure, stroke, ischemia, reperfusion, leishmaniasis, helminthiasis, hypoxia, or other causes of heart failure in a mammal, where increased cytokine activity is known to play a role. In a more particular embodiment, the invention relates to methods and compositions for detecting, in particular, ischemia, specifically silent ischemia, by obtaining a test sample from a mammal, measuring the level of IAP mRNA and or IAP protein, in the test sample, and determining if the level of IAP mRNA or protein measured in the test sample correlates with elevated levels indicative of ischemia.

The predetermined value for IAP can be established by one of at least two ways. For example, it can be established by gathering data from the mammal (e.g., a human) believed to be at risk of ischemia or reperfusion prior to the onset of signs for organ failure, or by testing other healthy mammals in preferably the same species and age group as the subject.

In the first method, the physician treating the subject may determine that the subject, based on statistical, genetic, familial or other factors generally known in the medicinal arts, is at risk of ischemia and/or reperfusion. The physician can then determine the level of IAP mRNA or protein expressed in appropriate cells and or media of the subject. Such procedures are well known in the art. In addition, the physician may also determine the level of secondary markers (e.g., TNF-alpha, IL-1, 2, 4, 6, 13, or another cytokine) to help quantify and establish an appropriate baseline for the individual or class. The methods of the invention provide for the comparison of the level of the IAP mRNA or protein and secondary marker(s) in the subject with this baseline, in order to detect impending organ failure, such as may be caused by ischemia, reperfusion or hypoxia.

In the second method, the physician or other health care professional (i.e., medical statistician), can determine the level of IAP mRNA or protein in individuals determined to be healthy. This may be done by routine procedures well known by those who practice the medical arts. The levels of the individuals in the same age group can be grouped together and their average and standard deviation determined. This value will represent the predetermined value to which the level of IAP mRNA or protein in the subject will be compared in order to detect ischemia, reperfusion or hypoxia.

Preferably, the level of IAP mRNA or protein detected in the practice of this invention is(are) different than a standard or reference measure that indicates a normal condition. More preferably, the level of the cardiac IAP mRNA or protein detected is greater than the standard measure. In certain embodiments, the level of the IAP mRNA or protein is measured quantitatively; in other embodiments, the measurement is semi-quantitative.

In preferred embodiments, the determination step of the method of invention is a comparison between the concentration of the IAP mRNA or protein and a predetermined value for the marker. In preferred embodiments, the predetermined value is indicative of a healthy condition. This predetermined value can be determined using the methods of the present invention as described above, and can be specific for a particular patient or generic for a given population. The predetermined value is preferably obtained from a mammal in the same species and approximately the same age as the mammal providing the test sample. In certain embodiments, the predetermined value may have been established by prior measurement of the particular patient's marker levels when the patient was healthy.

In a further aspect, the invention relates to a method of detecting organ failure (e.g., cardiac ischemia or hypoxia) in a mammal comprising the steps of (a) measuring a level of IAP mRNA or protein in a test sample from the mammal; and (b) determining if the level of the IAP mRNA or protein measured in the test sample correlates with ischemia, reperfusion or hypoxia. Such test samples can be obtained using methods well known to those of ordinary skill in the medicinal arts, including making a small puncture and removing the appropriate cells as is done routinely in oncology testing. Routine lab procedures can be used to quantify IAP mRNA and or protein in cells removed from the subject (see examples 1-4 for further details) and standardized kits and protocols can be used for measuring IAP mRNA or protein levels in blood, lymph, interstitial fluid, urine or other bodily fluid. For example, the R-5500A Purescript RNA whole blood purification system from Gentra Systems, Inc. or QIAamp RNA Blood mini kit from Qiagen, Inc. can be used to extract RNA from bodily cells and/or fluids and the RNA obtained can then be isolated, purified and tested using the RT-PCR methods.

In another aspect, the invention provides for a method of preventing or reducing the severity of a subsequent acute myocardial infarction (or other form of heart failure) by detecting ischemia, reperfusion or hypoxia, as described herein, and taking a preventive measure. The preventive measure is preferably selected from the group including but not hereby limited to blood transfusion, medication, hemofiltration, dialysis, coronary bypass surgery, preventive angioplasty, and/or administering therapeutically effective amounts of one or more anticoagulants, thrombolytics, or other pharmaceutical products intended to alleviate the ischemic or hypoxic condition. Furthermore, the methods of the invention allow a health care professional to determine the prognosis of a patient following a surgical procedure by detecting ischemia, reperfusion or hypoxia.

In preferred embodiments, the compositions of the invention further comprise a solid support to which the moiety detecting the IAP mRNA or protein is or can be attached. In certain embodiments, attachment of the detecting moiety, e.g. an antibody, nucleic acid or protein probe, is via a covalent linkage with the solid support. In other embodiments, attachment may be via a non-covalent linkage, for example, between members of a high affinity binding pair. Many examples of high affinity binding pairs are known in the art, and include biotin/avidin, ligand/receptor, and antigen/antibody pairs.

Vectors, Host Cells, and Expression

The present invention also relates to vectors that comprise a polynucleotide or polynucleotides of the present invention, and host cells that are genetically engineered with vectors of the invention and to the production of polypeptides of the invention by recombinant techniques. Cell-free translation systems can also be employed to produce such proteins using RNAs derived from the DNA constructs of the present invention.

For recombinant production, host cells can be genetically engineered to incorporate expression systems or portions thereof for polynucleotides of the present invention. Introduction of polynucleotides into host cells can be effected by methods described in many standard laboratory manuals, such as Davis et al., Basic Methods In molecular Biology (1986) and Sambrook et al., Molecular Cloning: A Laboratory Manual, 2nd Ed., Cold Spring Harbor Laboratory Press, Cold Spring Harbor, N.Y. (1989) such as calcium phosphate transfection, DEAE-dextran mediated transfection, transvection, microinjection, cationic lipid-mediated transfection, electroporation, transduction, scrape loading, ballistic introduction or infection.

Representative examples of appropriate hosts include bacterial cells, such as streptococci, staphylococci, E. coli, Streptomyces and Bacillus subtilis cells; fungal cells, such as yeast cells and Aspergiffits cells, insect cells such as Drosophila S2 and Spodoptera Sf9 cells; animal cells such as CHO, COS, HeLa, C127, 3T3, BHK, HEK 293 and Bowes melanoma cells, and plant cells. The selection of an appropriate host is deemed to be within the scope of those skilled in the art from the teachings herein.

More particularly, the present invention also includes recombinant constructs comprising the cDNA sequence. The constructs comprise a vector, such as a plasmid or viral vector, into which the clone has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available. The following vectors are provided by way of example; Bacterial: pQE70, pQE60, pQE-9 (Qiagen), pBS, pD10, phagescript, psiX 174, pbluescriot SK, pbsks, pNH8A, pNH 16a, pNHI8A, pNH46A (Stratagene); ptrc99a, pKK223-3, pKK233-3, pDR540, pRIT5 (Pharmacia); Eukaryotic: pWLNEO, pSV2CAT, pOG44, pXTI, pSG (Stratagene) pSVK3, pBPV, pMSG, pSVL (Pharmacia). As further examples, cDNA of human IAP may be inserted in the pEF/myc/cyto vector (from Invitrogen) and/or the pCMV-Tag3b vector (from Stratagene), which can then be used with anti-Myc Ab, to transform Hela (or other) cells with the IAP DNA. The protein produced may be purified from the cells and directly injected to the tissue, infused to blood cells, or delivered in a lypholized carrier as described above.

Further, recombinant IAP DNA, or polynucleotide sequences coding for the antisense sequence encoding the protein, may be directly injected to the organ for the production or inhibition of IAP endogenously. cDNA or polynucleotide sequences coding for the antisense sequence encoding the protein may also be delivered using the appropriate vectors, as described above, and well known in the recombinant arts. Such vectors, may also include adenovirus vector constructs, associated adenovirus vectors, and/or retrovirus vectors containing the appropriate promoters, gene insertion sites, termination sites, and the appropriate mutations so as to make them replication defective and safe for therapeutic uses.

However, any other plasmid or vector may be used as long as they are replicable and viable in the host. In addition, a complete mammalian transcription unit and a selectable marker can be inserted into a prokaryotic plasmid. The resulting vector is then amplified in bacteria before being transfected into cultured mammalian cells. Examples of vectors of this type include pTK2, pHyg and pRSVneo.

A great variety of expression systems can be used. Such systems include, among others, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia, viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector suitable to maintain, propagate or express polynucleotides to produce a polypeptide in a host may be used. The appropriate nucleotide sequence may be inserted into an expression system by any of a variety of well-known and routine techniques, such as, for example, those set forth in Sambrook et al., Molecular Cloning, A Laboratory Manual (supra).

Promoter regions can be selected from any desired gene using CAT (chloramphenicol acetyl transferase) vectors or other vectors with selectable markers. Two appropriate vectors are pKK232-8 and pCM7. Particular named bacterial promoters include lacI, lacZ, T3, T7, gpt, lambda PR, PL and trp. Eukaryotic promoters include CMV immediate early, HSV thymidine kinase, early and late SV40, LTRs from retrovirus, and mouse metallothionein-1. Selection of the appropriate vector and promoter is well within the level of ordinary skill in the art.

For secretion of the translated protein into the lumen of the endoplasmic reticulum, into the periplasmic space or into the extracellular environment, appropriate secretion signals may be incorporated into the desired polypeptide. These signals may be endogenous to the polypeptide or they may be heterologous signals.

If the IAP polypeptide is to be expressed for use in screening assays, generally, it is preferred that the polypeptide be produced at the surface of the cell. In this event, the cells may be harvested prior to use in the screening assay. If IAP polypeptide is secreted into the medium, the medium can be recovered in order to recover and purify the polypeptide; if produced intracellularly, the cells must first be lysed before the polypeptide is recovered. IAP polypeptides can be recovered and purified from recombinant cell cultures by well known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding proteins may be employed to regenerate active conformation when the polypeptide is denatured during isolation and or purification.

Antibodies

The present invention also provides antibodies capable of immunospecifically binding to polypeptides of the invention. Polyclonal or monoclonal antibodies directed towards the polypeptide encoded by IAP may be prepared according to standard methods. Monoclonal antibodies may be prepared according to general hybridoma methods of Kohler and Milstein, Nature (1975) 256:495-497), the trioma technique, the human B-cell hybridoma technique (Kozbor et al., Immunology Today (1983) 4:72) and the EBV-hybridoma technique (Cole et al., Monoclonal Antibodies And Cancer Therapy, pp. 77-96, Alan R. Liss, Inc., 1985). In a preferred embodiment, antibodies are prepared, which react immunospecifically with various epitopes of the IAP encoded polypeptides. These above-described antibodies may be employed to isolate or to identify clones expressing the polypeptide or to purify the polypeptides by affinity chromatography. Specific antibodies may be made in vivo using recombinant DNA and methods well know in the art.

Antibodies that are immunologically specific to IAP proteins, or specific epitopes thereof, may be utilized in affinity chromatography to isolate the IAP protein, to quantify the protein utilizing techniques such as western blotting and ELISA, or to immuno-precipitate IAP from a sample containing a mixture of proteins and other biological materials. The immuno-precipitation of IAP is particularly advantageous when utilized to isolate binding partners of IAP, as described above. Antibodies against IAP polypeptides may also be employed to treat diseases associated with the anti-inflammatory cytokine mediated immune response, namely, asthma, psoriasis, ischemia, reperfusion, stroke, among other inflammation-induced pathological conditions.

### EXAMPLES

The following description sets forth the general procedures involved in practicing the present invention. To the extent that specific materials are mentioned, it is merely for purposes of illustration and is not intended to limit the invention. Unless other-wise specified, general cloning procedures, such as those set forth in Sambrook et al., Molecular Cloning, supra or Ausubel et al. (eds) Current Protocols in Molecular Biology, John Wiley & Sons (2000) (hereinafter “Ausubel et al.”) are used.

### Example I

### Preparation of Nucleic Acids

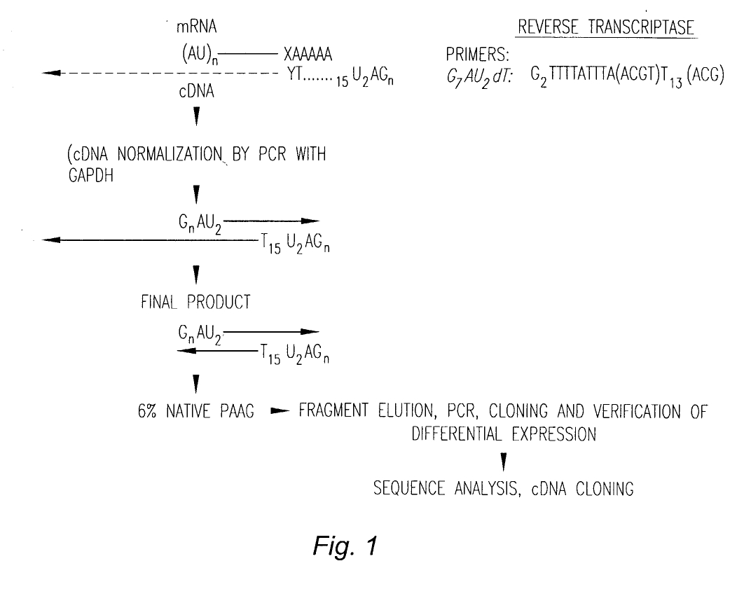

The IAP mouse protein coding region was cloned by targeted differential display method (TDD) (Dominguez, O., Ashhab, Y., Sabater, L., Belloso, E., Caro, P., Pujol-Borrell, R., 1998). A schematic representation of TDD is shown in FIG. 1. RNA extracted from mice subjected to kidney ischemia/reperfusion was used. A single reverse transcription with 200 U of Superscript II (Gibco BRL) and one primer (G₇AU₂dT, 1 μM) were used to produce cDNA using 2 μg of RNA extracted from kidneys of mice after sham- or I/R experiments. 20 ml of reaction mixture was incubated for 1 hr at 42° C. Reaction was finished by heating at 90° C. for 2 min and 1 U of RNAse H was added and incubated for 20 min at 37° C. To ensure comparability between normal and stressed cDNAs an initial assessment and normalization of the housekeeping gene, GAPDH was performed by PCR. The next step was a, PCR reaction with Dynazyme II polymerase and G₇TATTTATTTAT primer (3 μM), intended to anneal to ARE motifs, regardless of their immediate sequence context. Mixture contained four dNTPs at 0.2 mM and 0.25 μl of ³²P-dATP (NEN; 300 Ci/mmol). Reaction was performed with the following profile: 94° C. for 40 s, 42° C. for 1 min and 70° C. for 40 s. 35 cycles were applied. See FIG. 1, for more details.

Products were separated by electrophoresis in-native 6% polyacrylamide gels. At least 2 independent animals per each group were tested. Reactions are repeated at least 2 times each. After gels are fixed, dried and exposed, gel slices containing selected differentially expressed bands were cut out. DNA was eluted and used as a template for reamplification. After cloning the blunt-ended fragments into PCR Vector (Invitrogen), clones were sequenced, fragments isolated, labeled and used as a probes for Northern analysis to confirm differential expression.

Differential activation of the corresponding mRNA was confirmed by hybridization of the spotted on the filter DNA-sequence with probes obtained as a result of a new set of the differential display reaction using RNA from sham- or I/R-operated animals. To clone the full-sequence of the gene, Marathon cDNA amplification kit (Clontech) was used. 5′- and 3′-reactions were performed by using primers according to the manufacturer's protocol. Fragments obtained as a result of the final PCR reaction were cloned in PCR-TOPO vector (Invitrogen) and sequenced. Translation of the DNA sequence of SEQ ID NO:4 revealed existence of one open reading frame, coding the amino acid sequence of SEQ ID NO:5. Computer analysis of the protein sequence using PredictProtein software identified as a homologous sequence extracellular domain of the IL-13 alpha chain receptor. Computer analysis (BLAST search) of the non-redundant databases revealed presence of the highly homologous sequences in EST databases from human, rat, and bovine libraries.

### Example 2

### Mouse Kidney Ischemia/Reperfusion Model

Experimental procedure was as described earlier (Kelly, K. J., W. W. Williams, R. B. Colvin, M. Meehan, T. A. Spronger, J. Gutierrez-Ramos and J. V. Bonventre. 1997. Intracellular adhesion molecule-1-deficient mice are protected against ischemic and renal injury. J. Clin. Invest. 97:1056.). In brief, males weighing 25-35 g were anesthetized with sodium pentobarbital (25 mg/kg) and xylazine (10 mg/kg) and administered heparin (300 USP units per kg of body weight) subcutaneously prior to the surgery. Unilateral renal ischemia was induced by occluding the left side renal vein and artery with a microaneurysm clamp. Body temperature was maintained at 37° C. during the whole procedure. After 32 min of ischemia, the left kidney was reperfused by declamping the microaneurysm applicator and right nephrectomy was performed. Sham surgery consisted of a surgical procedure that was identical except that the mircoaneurysm clamp was not applied. Mice were sacrificed at different time points after surgery. Kidneys were removed immediately after perfusion with cold PBS and rapidly frozen in liquid nitrogen for RNA analysis.

### Example 3

### Preparation of RNA and Northern Blot Analysis

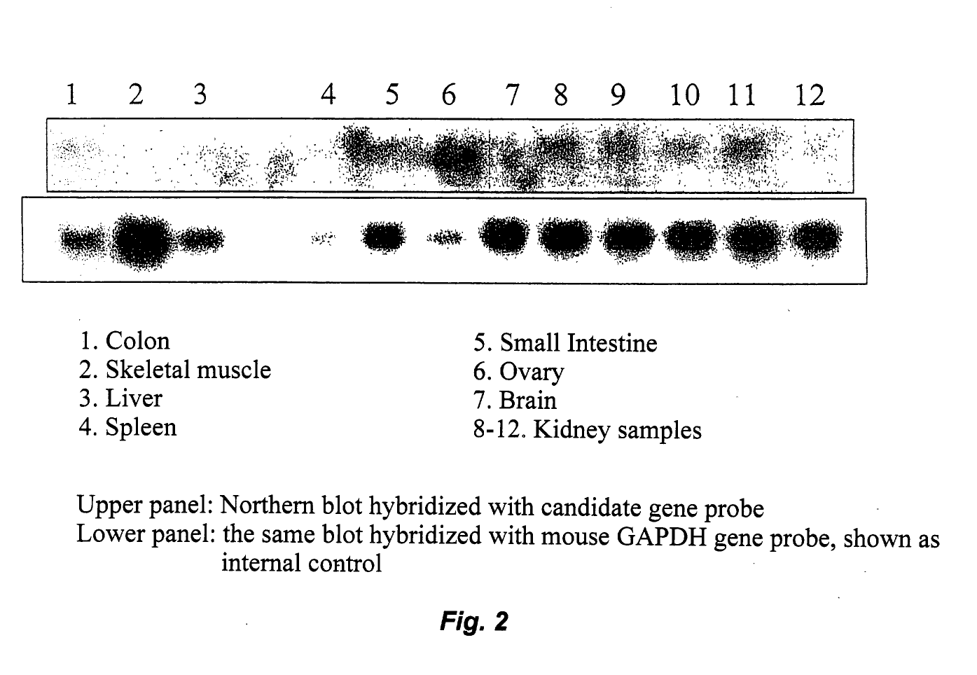

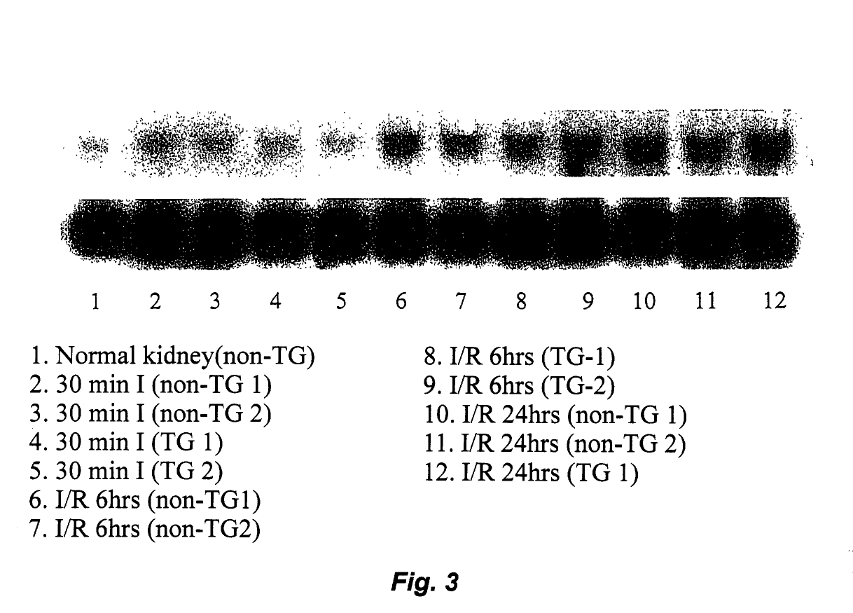

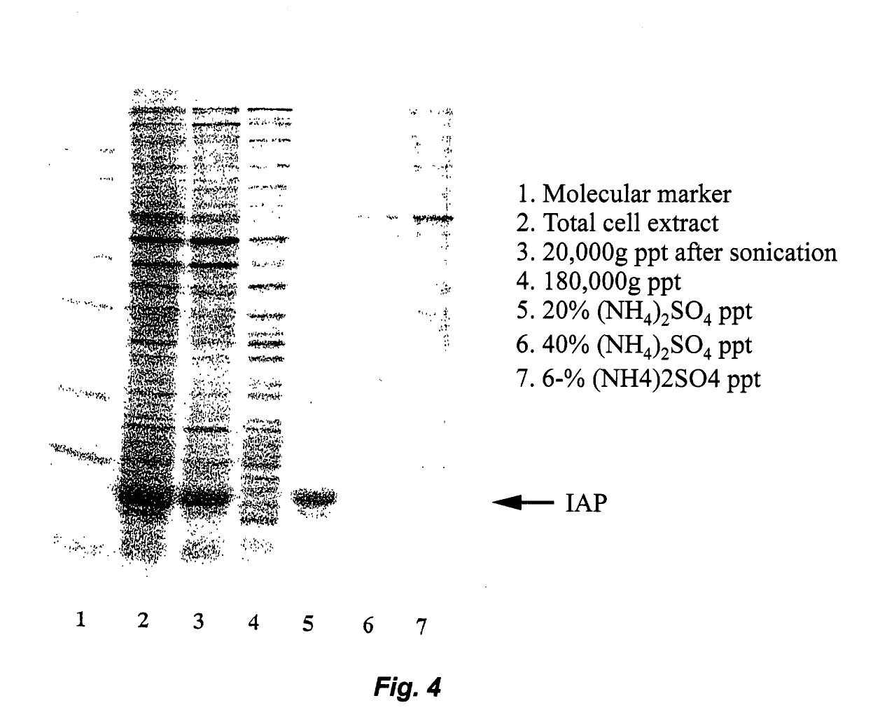

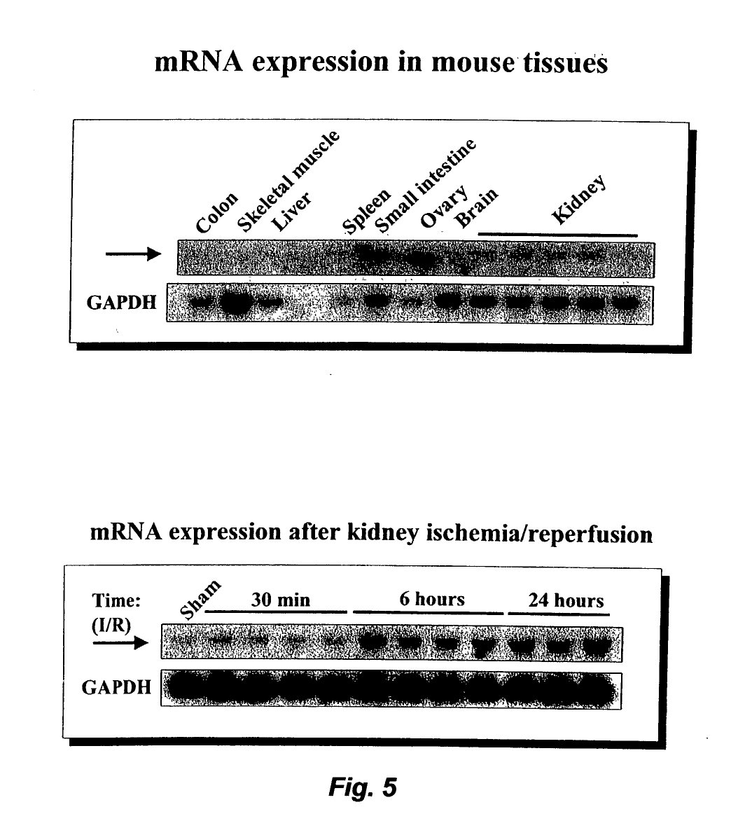

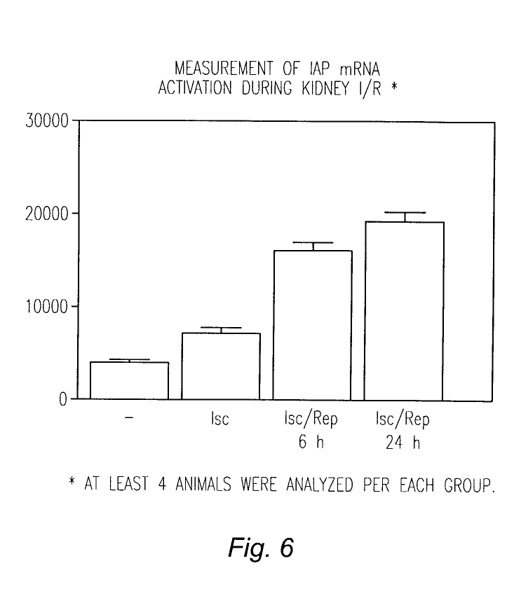

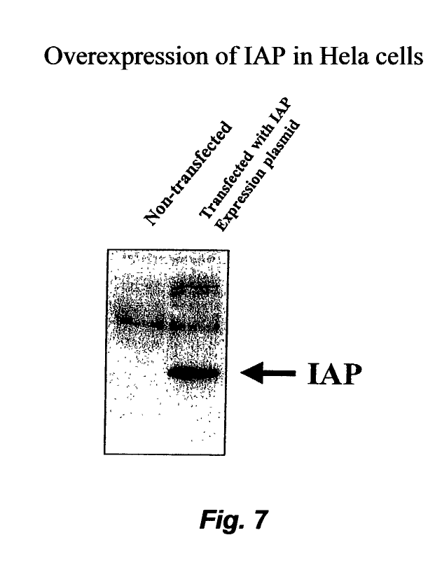

Total RNA was isolated from kidney samples using TRIzol reagent (GibcoBRL) according to manufacturer's protocol. The RNA concentration was measured by a spectrophotometer. For Northern blot analysis, RNA samples (20 μg) were loaded on 1.4% agarose gels with 2.2 M formaldehyde. After electrophoresis, RNA was transferred from the gels to GeneScreen™ membranes (NEN, Boston, Mass.) using a PosiBlot pressure blotter (Stratagene, La Jolla, Calif.). After transfer the membranes were fixed and hybridized with labeled DNA fragments at 42° C. overnight. As can be seen in comparing FIGS. 2 and 3, IAP mRNA expression is induced by the onset of ischemia/reperfusion, this can be seen in the intensity of the bands related to IAP mRNA expression at time periods wherein ischemia/reperfusion is evoked as compared to IAP mRNA expression at time periods wherein ischemia/reperfusion is not evoked. See also FIG. 5.

### Example 4

### Construction of Protein Expression System in E. coli

PCR amplification was conducted using the following primers: GGAATTCCATATGGAGCGCTCCGAGGAG (SEQ ID NO:10) and TAGGATCCTCAGGCAATGAGGCTTTG (SEQ ID NO:11). The YRACE product was cloned into a CR2.1 vector. The digested PCR product was inserted via a NdeI and BamH1 into bacterial expression vector pETII-a. E. coli BL21 cells were transformed with recombinant pET11-a plasmid that contains the protein coding sequence. Single transformants were inoculated into M9-CAA medium. Protein expression was induced by adding IPTG to mid-log phase culture. The induction time was 3 hours. The recombinant protein was extracted from cells and observed by electrophoresis in PAAG.

Human IAP may also be expressed as a fusion protein in E. coli using the pMAL system from Bio-Labs. Sequence coding for IAP is inserted downstream of the maltose-binding protein (malE) gene. Expression in JM83 hsdr cells is then induced by IPTG, as described above. This method allows for the production of soluble IAP proteins that may be purified in one step with an amylose column by high affinity chromatography. The protein can than be cleaved off from MBP by Factor Xa and purified by FPLC.

What is claimed is:

1. An isolated polynucleotide comprising a nucleotide sequence that has at least 70% identity to a nucleotide sequence encoding the IAP polypeptide of SEQ ID NO:2, said identity being over the entire region encoding SEQ ID NO:2.

2. The isolated polynucleotide of claim 1, comprising a nucleotide sequence that by virtue of redundancy of the genetic code, encodes for the amino acid sequence of SEQ ID NO:2.

3. An isolated polynucleotide, comprising a nucleotide sequence that has at least 70% identity to SEQ ID NO:1 or SEQ ID NO:3, said identity being calculated over the entire length of SEQ ID NO:1 or SEQ ID NO:3.

4. The polynucleotide of claim 3, comprising the nucleotide sequence of SEQ ID NO:1 or SEQ ID NO:3.

5. An expression vector comprising a polynucleotide, wherein said expression vector is capable of producing an IAP polypeptide comprising the amino acid sequence of SEQ ID NO:2 when said expression system is present in a compatible host cell.

6. A host cell comprising the expression system of claim 5.

7. A process for producing an IAP polypeptide comprising culturing a host of claim 6 under conditions sufficient for the production of said polypeptide and recovering the polypeptide from the culture.

8. A process for producing a cell which produces an IAP polypeptide comprising transforming, transducing or transfecting a host cell with the expression system of claim 5 such that the host cell, under appropriate culture conditions, produces an IAP polypeptide.

9. An isolated polypeptide comprising the amino acid sequence of SEQ ID NO:2.

10. An antibody immunospecific for the IAP polypeptide of claim 9.

11. An isolated polynucleotide comprising a nucleotide sequence that has at least 70% identity to a nucleotide sequence encoding the IAP polypeptide of SEQ ID NO:5, said identity being over the entire region encoding for SEQ ID NO:5.

12. The isolated polynucleotide of claim 11, comprising a nucleotide sequence that by virtue of redundancy of the genetic code, encodes for the amino acid sequence of SEQ ID NO:5.

13. An isolated polynucleotide, comprising a nucleotide sequence that has at least 70% identity to SEQ ID NO:4, said identity being calculated over the entire length of SEQ ID NO:4.

14. The isolated polynucleotide of claim 13, comprising the nucleotide sequence of SEQ ID NO:4.

15. An expression vector comprising a polynucleotide, wherein said expression vector is capable of producing an IAP polypeptide comprising the amino acid sequence of SEQ ID NO:5 when said expression system is present in a compatible host cell.

16. A host cell comprising the expression system of claim 15.

17. A process for producing a IAP polypeptide having the amino acid sequence of SEQ ID NO:5 comprising culturing a host of claim 16 under conditions sufficient for the production of said polypeptide and recovering the polypeptide from the culture.

18. A process for producing a cell which produces an IAP polypeptide, comprising transforming, transducing or transfecting a host cell with the expression system of claim 15 such that the host cell, under appropriate culture conditions, produces an IAP polypeptide having the amino acid sequence of SEQ ID NO:5.

19. An isolated polypeptide, comprising the amino acid sequence of SEQ ID NO:5.

20. An antibody immunospecific for the IAP polypeptide of claim 19.

21. An isolated polynucleotide, comprising a nucleotide sequence that has at least 70% identity to a nucleotide sequence encoding the IAP polypeptide of SEQ ID NO:7, said identity being over the entire region encoding for SEQ ID NO:7.

22. The isolated polynucleotide of claim 21, comprising a nucleotide sequence that by virtue of redundancy of the genetic code, encodes for the amino acid sequence of SEQ ID NO:7.

23. An isolated polynucleotide comprising a nucleotide sequence which has at least 70% identity to SEQ ID NO:6, said identity being calculated over the entire length of SEQ ID NO:6.

24. The isolated polynucleotide of claim 23, comprising the nucleotide sequence of SEQ ID NO:6.

25. An expression vector comprising a polynucleotide, wherein said expression vector is capable of producing an IAP polypeptide, comprising the amino acid sequence of SEQ ID NO:7 when said expression system is present in a compatible host cell.

26. A host cell comprising the expression system of claim 25.

27. A process for producing an IAP polypeptide having the amino acid sequence of SEQ ID NO:7 comprising culturing a host of claim 26 under conditions sufficient for the production of said polypeptide and recovering the polypeptide from the culture.

28. A process for producing a cell which produces an IAP polypeptide comprising transforming, transducing or transfecting a host cell with the expression system of claim 25 such that the host cell, under appropriate culture conditions, produces an IAP polypeptide having the amino acid sequence of SEQ ID NO:7.

29. An isolated polypeptide comprising the amino acid sequence of SEQ ID NO:7.

30. An antibody immunospecific for the IAP polypeptide of claim 29.

31. An isolated polynucleotide, comprising a nucleotide sequence that has at least 70% identity to a nucleotide sequence encoding the IAP polypeptide of SEQ ID NO:9, said identity being over the entire region encoding for SEQ ID NO:9.

32. The isolated polynucleotide of claim 31, comprising a nucleotide sequence which, by virtue of redundancy of the genetic code, encodes for the amino acid sequence of SEQ ID NO:9.

33. An isolated polynucleotide, comprising a nucleotide sequence that has at least 70% identity to SEQ ID NO:8, said identity being calculated over the entire length of SEQ ID NO:8.

34. The isolated polynucleotide of claim 33, comprising the nucleotide sequence of SEQ ID NO:8.

35. An expression vector comprising a polynucleotide, wherein said expression vector is capable of producing an IAP polypeptide comprising the amino acid sequence of SEQ ID NO:9 when said expression system is present in a compatible host cell.

36. A host cell comprising the expression system of claim 35.

37. A process for producing an IAP polypeptide having the amino acid sequence of SEQ ID NO:9 comprising culturing a host of claim 36 under conditions sufficient for the production of said polypeptide and recovering the polypeptide from the culture.

38. A process for producing a cell which produces an IAP polypeptide, comprising transforming, transducing or transfecting, a host cell with the expression system of claim 35 such that the host cell, under appropriate culture conditions, produces an IAP polypeptide having the amino acid sequence of SEQ ID NO:9.

39. An isolated polypeptide, comprising the amino acid sequence of SEQ ID NO:9.

40. An antibody immunospecific for the IAP polypeptide of claim 39.

41. An isolated polynucleotide encoding a soluble protein that binds IAP.

42. A method of detecting organ failure caused by ischemia, reperfusion or hypoxia in a mammal, the method comprising the steps of:
(a) measuring a level of a IAP mRNA in a test sample from said mammal; and
(b) determining if the level of the mRNA measured in said test sample correlates with an adverse organ condition in the mammal.

43. A method according to claim 42, wherein the adverse organ condition is a condition selected from the group consisting of renal, liver and heart failure.

44. A method according to claim 42, wherein the mammal is selected from the group consisting of human, mouse, rat and bovine.

45. A method according to claim 42, wherein said test sample is a cell removed from the body.

46. The method according to claim 45, wherein said cell is selected from the group consisting of kidney, liver and heart cells.

47. A method according to claim 42, wherein said test sample is a body fluid.

48. A method according to claim 47, wherein said body fluid is selected from the group consisting of blood, interstitial fluid, urine and lymph.

49. A method according to claim 47 wherein said body fluid is blood.

50. A method according to claim 42, wherein said level of said IAP mRNA is a concentration of said IAP mRNA.

51. A method according to claim 42, wherein said measuring step comprises measuring said IAP mRNA level by a method selected from the group consisting of chromatography, immunoassay, enzymatic assay, spectroscopy, and Southern and Northern blot assays.

52. A method according to claim 51, wherein said measuring is a chromatographic method selected from the group consisting of high performance liquid chromatography and gas chromatography.

53. A method according to claim 51, wherein said measuring is a spectroscopic method selected from the group consisting of ultraviolet spectroscopy, infrared spectroscopy and nuclear magnetic resonance spectroscopy.

54. A method according to claim 51, wherein said measuring is an immunoassay that detects an IAP protein coded for by the IAP mRNA of claim 1 in said test sample using anti-IAP antibodies.

55. A method according to claim 42, wherein said determination step is a comparison between said measured level of said IAP mRNA and a predetermined value for the level of said IAP mRNA.

56. A method according to claim 55, wherein said predetermined value for the level of the IAP mRNA is indicative of normal function.

57. A method according to claim 56, wherein said predetermined value for the level of said IAP mRNA is obtained from a mammal of the same species and approximately the same age as the mammal from which the test sample was obtained.

58. A method according to claim 57, wherein said measured level of said IAP mRNA differs from said predetermined value for the level of said IAP mRNA.

59. A method of preventing organ failure in a mammal by detecting the onset of ischemia, reperfusion or hypoxia according to claim 42 and taking a preventive measure.

60. A method of determining prognosis of a patient following a medical procedure by detecting ischemia, reperfusion or hypoxia according to claim 42.

61. A method according to claim 60, wherein said medical procedure is selected from the group consisting of organ transplantation, coronary bypass surgery, angioplasty, and administering an anticoagulant.

62. A method of detecting organ failure caused by ischemia, reperfusion or hypoxia in a mammal, the method comprising the steps of:
(a) measuring a level of an IPA protein in a test sample from said mammal; and
(b) determining if the level of said IAP protein measured in said test sample correlates with an adverse organ condition.

63. A method according to claim 62, wherein said adverse organ condition is a condition selected from the group consisting of renal, liver and heart failure.

64. A method according to claim 62, wherein the mammal is selected from the group consisting of human, mouse, rat and bovine.

65. A method according to claim 62, wherein said test sample is a cell removed from the body.

66. The method according to claim 65, wherein said cell is selected from the group consisting of kidney, liver, and heart cells.

67. A method according to claim 62, wherein said test sample is a body fluid.

68. A method according to claim 67, wherein said body fluid is selected from the group consisting of blood, interstitial fluid, urine and lymph.

69. A method according to claim 67 wherein said body fluid is blood.

70. A method according to claim 62, wherein said level of said IAP protein is a concentration of said IAP protein.

71. A method according to claim 62, wherein said measuring step comprises measuring said IAP protein level by a method selected from the group consisting of chromatography, immunoassay, enzymatic assay, spectroscopy, and Western blot assays.

72. A method according to claim 71, wherein said measuring is a chromatographic method selected from the group consisting of high performance liquid chromatography and gas chromatography.

73. A method according to claim 71, wherein said measuring is a spectroscopic method selected from the group consisting of ultraviolet spectroscopy, infrared spectroscopy and nuclear magnetic resonance spectroscopy.

74. A method according to claim 71, wherein said measuring is an immunoassay that detects an IAP protein using anti-IAP antibodies.

75. A method according to claim 62, wherein said determination step is a comparison between said measured level of said IAP protein and a predetermined value for the level of said IAP protein.

76. A method according to claim 75, wherein said predetermined value for the level of the IAP protein is indicative of normal cardiac function.

77. A method according to claim 76, wherein said predetermined value for the level of said IAP protein is obtained from a mammal of the same species and approximately the same age as the mammal from which the test sample was obtained.

78. A method according to claim 77, wherein said measured level of said IAP protein differs from said predetermined value for the level of said IAP protein.

79. A method of preventing an organ failure in a mammal by detecting ischemia, reperfusion or hypoxia according to claim 62 and taking a preventive measure.

80. A method of determining prognosis of a patient following a medical procedure by detecting ischemia, reperfusion or hypoxia according to claim 62.

81. A method according to claim 80, wherein said medical procedure is selected from the group consisting of organ transplantation, coronary bypass surgery, angioplasty, and administering an anticoagulant.

82. A method of detecting a condition that is related to an increased inflammatory response in a mammal, the method comprising the steps of:
(a) measuring a level of an IPA mRNA or protein in a test sample from said mammal; and
(b) determining if the level of said IAP mRNA protein measured in said test sample correlates with an adverse inflammatory condition.

83. A method according to claim 62, wherein said adverse organ condition is septic shock.


SEQUENCE LISTING (8 sequences)

SEQ ID NO: 1 - DNA, 840 bp, Homo sapiens
atgtctccggcgcgtcggtgcagggggatgagggccgcggtggctgccagcgtggggttgagcgaggggcctgctggctcccggagcggtcgcctcttccgcccgccgagtcccgctccggcggcccccggcgcccggctgttgcggctcccggggagcggggccgtgcaggccgcgagcccggagcgcgccggctggaccgaggcgctgcgggccgccgtggccgagctgcgcgccggcgccgtggtggccgtccccaccgatacgctgtacggcctggcctgcgcggcgagctgctcggcggctctgcgcgctgtgtaccgcctcaagggtcgcagcgaggccaagcctctggccgtatgcctcggccgcgtggccgacgtctacagatactgccgtgtgagagtacctgaggggctcctgaaagacctactgccaggaccagtgaccctggtgatggaacgctcggaggagctcaacaaggacctaaacccttttacgcctcttgtaggcattcggattcctgatcatgcttttatgcaagacttggctcagatgtttgagggtccgcttgctctcactagtgccaacctcagctcccaggccagttctctgaatgtcgaggagttccaggatctctggcctcagttgtccttggttattgatgggggacaaattggggatggccagagccccgagtgtcgccttggctcaactgtggttgatttgtctgtgcccggaaagtttggcatcattcgtccaggctgtgccctggaaagtactacagccatcctccaacagaagtacggactgctcccctcacatgcgtcctacctgtga

SEQ ID NO: 2 - protein, 279 aa, Homo sapiens
MSPARRCRGMRAAVAASVGLSEGPAGSRSGRLFRPPSPAPAAPGARLLRLPGSGAVQAASPERAGYTEALRAAVAELRAGAVVAVPTDTLYGLACAASCSAALRAVYRLKGRSEAKPLAVCLGRVADVYRYCRVRVPEGLLKDLLPGPVTLVMERSEELNKDLNPFTPLVGIRIPDHAFMQDLAQMFEGPLALTSANLSSQASSLNVEEFQDLYPQLSLVIDGGQIGDGQSPECRLGSTVVDLSVPGKFGIIRPGCALESTTAILQQKYGLLPSHASYL

SEQ ID NO: 3 - DNA, 1387 bp, Homo sapiens
  misc_feature (1)..(1387): The letter “n” stands for a substitution base.
natttcggcactagggaacgctcggaggagctcaacaaggacctaaacccttttacgcctcttgtaggcattcggattcctgatcatgcttttatgcaagacttggctcagatgtttgagggtccgcttgctctcactagtgccaacctcagctcccaggccagttctctgaatgtcgaggagttccaggatctctggcctcagttgtccttggttattgatgggggacaaattggggatggccagagccccgagtgtcgccttggctcaactgtggttgatttgtctgtgcccggaaagtttggcatcattcgtccagggtgtgcctgggaaagtactacagccatcctccaacagaagtacggactgctcccctcacatgcgtcctacctgtgaaactctgggaagcaggaaggcccaagacctggtgctggatactatgtgtctgtccactgacgactgtcaaggcctcatttgcagaggccaccggagctagggcactagcctgacttttaaggcagtgtgtctttctgagcactgtagaccaagcccttggagctgctggtttagccttgcacctggggaaaggatgtatttatttgtattttcatatatcagccaaaagctgaatggaaaagttaagaacattcctaggtggccttattctaataagtttcttctgtctgttttgtttttcaattgaaaagtaattaaataacagatttagaatctagtgagagcntcctctctgggggtggtggcatttaaggttcaacccanccnagaagtgctgcgctgtttaaaaagtctcaggtggctgcgtgtggtggctcatgcctgtaatcccaacattctgggaggcccaggcgggagaactgcttgagcccaggagttcagaatcagcctgggcaacatagcaatactccgtctcataaaaattaataaataaaaagtctcaggtgaccaaaggctcctgaagctagaaccaggtttggataaagattgaagagccacaggccactcttccctctgagccattgggcctagtggtgtcatgtattgtaattgctcgcagggagagcagtctttttggtgtaatagtgggatgtctgcttagttggcaggggttcagtccaaatggaagaatattgggaaataaacctccnctatcctttatagccagggacttttttcttatttattcataaaataaattatagttaattatacccataacacctttatttaaatccagtgttctccgcagccttttgtctatttatatgtgtaccaagtgttaaacataattattattgggcatttgaacntgtttttcnttanaganatnctgnnattaaacatatttgtnaatggnaaaaaaaaaaaa

SEQ ID NO: 4 - DNA, 930 bp, Mus musculus
atgtctacggcgcgtccgtgcgcggggctgagggccgccgtggcggccggcatggggttgagcgacgggccggctagttctggccgcggctgccgcctcctactccctcctgagcccgctccggcgctgccgggggcccggctgctgcggcttccggagagcgagcccgtggaagccgcgagccccgagcgcgccggctggaccgaggcgctgcgggccgccgtggccgagctgcgcgccggcgccgtggtggcggtcccgaccgacacgctctacggcctggcctgctcggcgagcagctcggcggccctgagttgcgtgtaccgcctcaaaggccgcagcgaggccaagccgctggccgtgtgcctgggccgcgtggccgacgtctacaggtactgtcaggtgagagtacctagggagctcctggaagacctgttcccaggccctgtgaccctggtgatggagcgctccgaggagctcaacaaagacctgaacccctttactcgtcttgttggcatccggattcctgaccatgccttcatgctggacttggcccagatgtttgggggaccacttgcactcactagtgccaacctcagctcccaggccagttctctgagtgttgaggagttccaagacctctggcctcatttgtcccttgtcattgatggggggccaattggggatagtcagagccctgagtgtcgcctcggctctactgtggttgacttatctgtgcctggaaagtttggcattattcgcccaggctgtgccctggaaaacactacatcgatcctccagcagaaatatgggctgctcccttcacaggggtcctgttcatgaaacttgggaggacccaaggacatgctggatactatgtgtctgctactggatgcaaagcctcattgcctgaggttcctacatctatag

SEQ ID NO: 5 - protein, 280 aa, Mus musculus
MSTARPCAGLRAAVAAGMGLSDGPASSGRGCRLLLPPEPAPALPGARLLRLPESEPVEAASPERAGYTEALRAAVAELRAGAVVAVPTDTLYGLACSASSSAALSCVYRLKGRSEAKPLAVCLGRVADVYRYCQVRVPRELLEDLFPGPVTLVMERSEELNKDLNPFTRLVGIRIPDHAFMLDLAQMFGGPLALTSANLSSQASSLSVEEFQDLYPHLSLVIDGGPIGDSQSPECRLGSTVVDLSVPGKFGIIRPGCALENTTSILQQKYGLLPSQGSCS

SEQ ID NO: 6 - DNA, 702 bp, Bos taurus
ggccgtccccaacgatacgctgtacgggctggcctgctcggcgagctgctcggaagcactgggcgccgtgtaccgtgtcaagggccgcagcgagaccaagccgctggccgtatgcctgggccgcgtggccgacgtctacaggtactgccacgtgagagtacctgaggggctcctgaaggacctgttgccaggaccagtgaccctggtgatggaacgctcagaggagctcaacaaggacctgaatcctttcactcctcttgtaggcatccggattcctgaccacgccttcatgcaggacttggtccagatgtttggggggccactcgctctcaccagtgccaacctcagctcccagtccagctctctgaatgttgaggaattccaggacctgtggcctcacttgtccctgatcattggtgggggaccaattggggacggccagagcccagagtgtcgactaggctcaactgtggttgacttgtctgtgcctggaaagtttggcatcattcgtcctggttgtgcccttgaaagtacttcagccatcctccaggagtatgggctgctcccctcacatggatcctgctggtgacactctggaggagggaaggcccaagggctggtgctggacactatgtgtccgactgctggtggttggcaaggcctcatttgcagaggctgctagggctacagtgttagtagtct

SEQ ID NO: 7 - protein, 126 aa, Bos taurus
MERSEELNKDLNPFTPLVGIRIPDHAFMQDLVQMFGGPLALTSANLSSQSSSLNVEEFQDLWPHLSLIIGGGPIGDGQSPECRLGSTVVDLSVPGKFGIIRPGCALESTSAILQEYGLLPSHGSCW

SEQ ID NO: 8 - DNA, 841 bp, Rattus novartis
  misc_feature (491)..(491): The letter “z” stands for sequence hybridizing.
gatagtgaaagccctgagtgtcgtcttggctctactgtggttgacttgtctgtgcctggaaagtttggcattattcgctcaggctgtgccctggaaaatactacagccatcctccaggggaaatatgggctgctcccttcacaggggtcctgttcatgaaacttgggaggacccaagaaccatgctggatactatgtgtctactacaggttggcaaagcctcattggctgaggttcctggagctacatctgtagcctagctttttaggcagtgtccttggctctgaatcctgtaggccagccagaagcttcgggttgagccttgcacccaggggaaggttatatttactctgtagattcatgtgtcaacccagaatggagggaagaacattcttagagtgaccttattattttaagtgcccctctcaccccaaccctgcctataagttaagtaacttgactgcagaattagaatgcattaagagctgcttactggtgaacagtgaaatttggtttaaaaccagccagaagcactaatgcagtctagaagtctcaggaccaatgcagcaaagtctaggagccctggccagagctttctgggtacaggagagtggtcatttggagaaaattattctaggagttccaaatgaaataatattgaaaaataaaatcttgactgttttcagccagtgactttcttatttattggtatagttctctgtttaatttatttaactcagaagtcatctttgttcatatgtctacctggtatttacataattatttttaagtatttgaactgtatttctttattaaatatttcttctacaaaaaaaaaaaaa